SEQUENCE LISTING (24 sequences)

SEQ ID NO: 1 - protein, 868 aa, Homo sapiens
MASFPETDFQICLLCKEMCGSPAPLSSNSSASSSSSQTSTSSGGGGGGPGAAARRLHVLPCLHAFCRPCLEAHRLPAAGGGAAGEPLKLRCPVCDQKVVLAEAAGMDALPSSAFLLSNLLDAVVATADEPPPKNGRAGAPAGAGGHSNHRHHAHHAHPRASASAPPLPQAPQPPAPSRSAPGGPAASPSALLLRRPHGCSSCDEGNAASSRCLDCQEHLCDNCVRAHQRVRLTKDHYIERGPPGPGAAAAAQQLGLGPPFPGPPFSILSVFPERLGFCQHHDDEVLHLYCDTCSVPICRECTMGRHGGHSFIYLQEALQDSRALTIQLLADAQQGRQAIQLSIEQAQTVAEQVEMKAKVVQSEVKAVTARHKKALEERECELLWKVEKIRQVKAKSLYLQVEKLRQNLNKLESTISAVQQVLEEGRALDILLARDRMLAQVQELKTVRSLLQPQEDDRVMFTPPDQALYLAIKSFGFVSSGAFAPLTKATGDGLKRALQGKVASFTVIGYDHDGEPRLSGGDLMSAVVLGPDGNLFGAEVSDQQNGTYVVSYRPQLEGEHLVSVTLCNQHIENSPFKVVVKSGRSYVGIGLPGLSFGSEGDSDGKLCRPWGVSVDKEGYIIVADRSNNRIQVFKPCGAFHHKFGTLGSRPGQFDRPAGVACDASRRIVVADKDNHRIQIFTFEGQFLLKFGEKGTKNGQFNYPWDVAVNSEGKILVSDTRNHRIQLFGPDGVFLNKYGFEGALWKHFDSPRGVAFNHEGHLVVTDFNNHRLLVIHPDCQSARFLGSEGTGNGQFLRPQGVAVDQEGRIIVADSRNHRVQMFESNGSFLCKFGAQGSGFGQMDRPSGIAITPDGMIVVVDFGNNRILVF

SEQ ID NO: 2 - DNA, 3138 bp, homo sapiens
gctctctcctcctcctcctcctcttcctctctggtctcctccctcctccgggctgggttgcaaatggcttcgttccccgagaccgatttccagatctgcttgctgtgcaaggagatgtgcggctcgccggcgccgctctcctccaactcgtccgcgtcgtcgtcctcctcgcagacgtccacgtcgtcggggggcggcggcgggggccctggggcggcggcgcgccgcctacacgtcctgccctgcctgcacgccttctgccgcccctgcctcgaggcgcaccggctgccggcggcgggcggcggcgcggcgggagagccgctcaagctgcgctgccccgtgtgcgaccagaaagtagtgctagccgaggcggcgggtatggacgcgctgccttcgtccgccttcctgcttagcaacctgctcgacgcggtggtggccactgccgacgagccgccgcccaagaacgggcgcgccggcgctccggcgggagcgggcggccacagcaaccaccggcaccacgctcaccacgcgcacccgcgcgcgtccgcctccgcgccgccactcccgcaggcgccgcagccgcccgcgccttcccgctcggcacccggcggccctgccgcttccccgtcggcgctgctgctccgccgtcctcacggctgcagctcgtgcgatgagggcaacgcagcttcttcgcgctgcctcgactgccaggagcacctgtgcgacaactgcgtccgagcgcaccagcgcgtgcgcctcaccaaggaccactacatcgagcgcggcccgccgggtcccggtgccgcagcagcggcgcagcagctcgggctcgggccgccctttcccggcccgcccttctccatcctctcagtgtttcccgagcgcctcggcttctgccagcaccacgacgacgaggtgctgcacctgtactgtgacacttgctctgtacccatctgtcgtgagtgcacaatgggccggcatgggggccacagcttcatctacctccaggaggcactgcaggactcacgggcactcaccatccagctgctggcagatgcccagcagggacgacaggcaatccagctgagcatcgagcaggcccagacggtggcggaacaggtggagatgaaggcgaaggttgtgcagtcggaggtcaaagccgtgacggcgaggcataagaaagccctggaggaacgcgagtgtgagctgctgtggaaggtagaaaagatccgccaggtgaaagccaagtctctgtacctgcaggtggagaagctgcggcaaaacctcaacaagcttgagagcaccatcagtgccgtgcagcaggtcctggaggagggtagagcgctagacatcctactggcccgagaccggatgctggcccaggtgcaggagctgaagaccgtgcggagcctcctgcagccccaggaagacgaccgagtcatgttcacaccccccgatcaggcactgtaccttgccatcaagtcttttggctttgttagcagcggggcctttgccccactcaccaaggccacaggcgatggcctcaagcgtgccctccagggtaaggtggcctccttcacagtcattggttatgaccacgatggtgagccccgcctctcaggaggcgacctgatgtcggctgtggtcctgggccctgatggcaacctgtttggtgcagaggtgagtgatcagcagaatgggacatacgtggtgagttaccgaccccagctggagggtgagcacctggtatctgtgacactgtgcaaccagcacattgagaacagccctttcaaggtggtggtcaagtcaggccgcagctacgtgggcattgggctcccgggcctgagcttcggcagtgagggtgacagcgatggcaagctctgccgcccttggggtgtgagtgtagacaaggagggctacatcattgtcgccgaccgcagcaacaaccgcatccaggtgttcaagccctgcggcgccttccaccacaaattcggcaccctgggctcccggcctgggcagttcgaccgaccagccggcgtggcctgtgacgcctcacgcaggatcgtggtggctgacaaggacaatcatcgcatccagatcttcacgttcgagggccagttcctcctcaagtttggtgagaaaggaaccaagaatgggcagttcaactacccttgggatgtggcggtgaattctgagggcaagatcctggtctcagacacgaggaaccaccggatccagctgtttgggcctgatggtgtcttcctaaacaagtatggcttcgagggggctctctggaagcactttgactccccacggggtgtggccttcaaccatgagggccacttggtggtcactgacttcaacaaccaccggctcctggttattcaccccgactgccagtcggcacgctttctgggctcggagggcacaggcaatgggcagttcctgcgcccacaaggggtagctgtggaccaggaagggcgcatcattgtggcggattccaggaaccatcgggtacagatgtttgaatccaacggcagcttcctgtgcaagtttggtgctcaaggcagcggctttgggcagatggaccgcccttccggcatcgccatcacccccgacggaatgatcgttgtggtggactttggcaacaatcgaatcctcgtcttctaattgcatttcctaggtttctgtgtttggggtgtgtgtgcgtgtctctctctctctctctctctttctctttctctctctttttgaatttcaaagaagaaacagtctcagggaaatttcttttttcttttttttttttaaagagaacaagaaaagtacaacattgcttaagtcctacctcatctttatttttttacagatgaatgtacttatcttttctgcagggattgagcctgtgaagtgataatttctatctacctcataaatctttacatttccttctgcaacaggccctcttcccctcctcagtggagtttgcatttccctcttcccctgcgtggggcatgatatgcacaagcctggcatctgtatggctgggagggcactggatgtgtgtggtggggtgtattctgtagattgagccaaggaaacacaaaaaaaaactactaagtaaaaaaacaaaaaactata

SEQ ID NO: 3 - protein, 855 aa, Mus musculus
MASFPETDFQICLLCKEMCGSPAPLSSNSSASSSSSQTSTSSAGGGGPGAAARRLHVLPCLHAFCRPCLEAHRLPAPGGAGPAEALKLRCPVCDQKVVLAEAAGMDALPSSAFLLSNLLDAVVATAEEPPPKNGRAGGGPGGAGGHSNHRHHAHHPAQRAAAPAPQPPPGPAASPGSLLMRRPHGCSSCDEGNAASSRCLDCQEHLCDNCVRAHQRVRLTKDHYIERGPPGPAAASAAQQLGLGPPFAGAPFSILSVFPERLGFCQHHDDEVLHLYCDTCSVPICRECTLGRHGGHSFAYLQDALQDSRALTIQLLADAQQGRQALQLSIEQAQTVAEQVEMKAKVVQSEVKAVTARHKKALEDRECELLWKVEKIRQVKAKSLFLQVEKLRQSLSKLESTISAVQQVLEEGRALDILLARDRMLAQVQELKTIRGLLQPQEDDRIMFTPPDQALYLALKSIGFVSSGAFAPLTKATGDGIKRALQGKVASFTVMGYDHDGEPRHSGGDLMSVVVLGPDGNLFGAEVSDQQNGTYIVSYRPQLEGEHLVSVTLYNQHIENSPFKVVVKSGRSYVGIGLPGLSFGSEGDGEGKLCRPWGVSVDKEGFIIVADRSNNRIQVFKPCGSFHHKFGTLGSRPGQFDRPAGVACDASRRIIVADKDNHRIQIFTFEGQFLLKFGEKGTKNGQFNYPWDVAVNSEGKILVSDTRNHRIQLFGPDGVFLNKYGFEGSLWKHFDSPRGVAFNNEGHLVVTDFNNHRLLVIHPDCQSARFLGSEGSGNGQFLRPQGVAVDQEGRIIVADSRNHRVQMFEANGSFLCKFGAQGSGFGQMDRPSGIAVTPDGLIVVVDFGNNRILIF

SEQ ID NO: 4 - DNA, 5186 bp, Mus musculus
gtttgctcctttggagattaaaaggattccgaattccgatcagtcccaataacccgcgttctgagaggcggcaaagcggagaaagaagggggacacgcgcacgcacacagcccctcgttcgggggagtcgtgcaaaataaagcccaaagcaacccaggacaccagggcatcgggagagcctgagcccggtaggcgcagcgcagccggggccacccgcatccccgctcggcgaatcgttcgtaggaggatgacgcaggcgggagccgcgggcggcgttggccacgcggctcccgaagtggccgcgcgggtccctcgcgtactcctctgattcgagcaagcccgggagggagactcggttcccgaggctccgggattgaaggggggacccaagcgagggtgaaatggcccgatgacaaaggaaatgtgatcctccttcgctgcccaggtttcggagaggacggccgtgaggaggaggggcgcgcggctcgcgcggaacgcggatagcattgcacgacggccccgcgcggagcgggcggcccgtacatccaatatcttcccgccgatgaataattcagggcggcccgcggggacctgcggccccgcgccgcggagcgcctgaggccggagaagggtcggagcgcgcgctagagagcgagccaggaggccggaggacacgcgggcgccgggccgcgtggggcgcgcgcgcgtgtgagcgcatagagaccgcggcccggcagggctgcgcacgggcgcggagcgtgtggggcgcgcgtgcgtacttggcacgcgtgcttccgcgggccgggggcccccgcacgaggcgcagctgcgcgcccgagccgaccgcgccgcgggggaggctgcgcctcggccgccacgcaggagctcctgccgccgcgtcatgcggcggggcggccggcggcgcgcgcggggacttggactgctgcgactcccggacgttttcccctccctccaggaggctcaattcatttagccggagggcaaggaaaaaaaaaaacaacacaagcaagactcgttgggaccaaattggtttgcgccgcggggaccggattccacggcggactttgagacttgtgcgggcgctcgtttgctgaggagtagcagtgcaaacccgccctgccgaggtggcccaggggtcgcctccaccgcgtcctcgatgcgctctgctgagtgagccgcgcgccctcatcctcctcgccgcgcgccctctgccctccagcccgcctgcaaatggcttcgttcccggagaccgacttccagatctgcctgctgtgcaaggagatgtgcggctcgcccgcgccgctctcctccaactcgtccgcctcgtcgtcgtcctcgcagacttccacgtcgtcggcgggcggcggcggccccggggcggcggcgcggcgtcttcacgtcttgccctgcctgcacgccttctgccggccgtgcctcgaggcgcaccggctgcccgctcccggaggcgcgggccccgccgaggcgctgaagctgcgctgccccgtatgtgaccagaaggtggtcctggccgaggcggcgggcatggacgcgctgccatcctccgccttcctgctcagcaacctgctggacgccgtggtggccaccgccgaagagccgccgcccaagaacgggcgcgcgggcggcggccccgggggcgcgggcggccacagcaaccaccggcaccatgcgcaccacccggcccagcgcgccgccgctccagcgccgcagccgccgcccggcccggccgcctcccccgggtcgctgctgatgcgccggccgcacggctgcagctcctgcgacgagggcaacgccgcctcgtcgcgctgcctcgactgccaggaacacctgtgcgacaactgtgtccgcgcgcaccagcgcgtccgcctcaccaaggaccattacatcgagcgcggccctccggggcctgcggccgcctccgcggcacagcagctggggctcgggccgcccttcgcgggcgcgcccttctccattctctcggtgttcccggagcgcctcggcttctgccagcaccacgacgatgaggtcctgcatctctactgtgacacctgctctgtccccatctgcagagagtgcacgctgggccgccacggtggccacagcttcgcctacctccaggatgccctgcaggactctcgggcactcaccatccagctgctggctgatgcccagcagggccgccaggctctccagctgagcatcgagcaggcccagactgtggcagaacaggtggagatgaaggcgaaggtggtccagtctgaggtcaaggccgtcacagctcgccacaagaaggccctggaggatcgggagtgtgagctgttgtggaaggtggagaagatccggcaggtgaaggcgaagtctctgttcctgcaggtggagaagctgcggcagagtctcagcaagctggagagcaccatcagcgccgtgcagcaggtcctggaagagggccgcgccctggacatcctgctggcccgagaccggatgctggcccaggtgcaggagctcaagaccatccgaggcctcctgcagccccaagaagatgaccgcattatgttcacgccccctgaccaggccctgtacctcgccctcaagtctattggctttgtcagcagcggggccttcgccccgctcacgaaggctacaggagatggcatcaagcgtgcccttcaggggaaggtggcctctttcactgtcatgggctacgaccacgatggtgagccccgccattcgggaggtgacctgatgtcagttgtggtcctcggccccgacggaaacctgtttggtgcagaagtgagcgaccagcagaatggtacttacatagtgagctacaggccccagcttgagggtgagcacttggtgtcggtgacattgtacaatcagcatatagagaacagccccttcaaggtcgtggtcaagtcgggccgcagctacgtgggcatcgggctccccgggctgagcttcggcagtgaaggtgacggggagggcaagctctgcaggccctggggcgtgagtgttgataaggagggtttcatcatagtggctgaccgaagcaataacagaatccaggtgtttaagccctgtggttccttccaccacaagtttggtacccttgggtctcgtcctgggcagtttgaccgaccggctggggtggcttgtgacgcctcccgcaggatcatagtggccgacaaagacaatcatcgcattcagatcttcaccttcgagggccagttccttcttaagtttggtgagaaaggcaccaagaacgggcagtttaactacccgtgggatgtagcagtgaattctgagggcaagatcctcgtttcagacacccggaatcatcgcatccagttgttcggaccggacggggtcttcctgaacaagtacggcttcgagggatctctctggaagcacttcgactctccacggggcgtggctttcaacaatgaaggtcacctggtagtcaccgacttcaacaaccaccggcttctggttattcaccctgattgccaatctgcccgcttcttgggttcggagggctcgggcaatgggcagttcctgcgaccacagggtgtggctgtggaccaggaagggagaatcatcgtagccgattccagaaatcaccgcgtgcagatgtttgaggccaatggcagcttcctgtgcaagtttggtgctcaagggagtggcttcgggcagatggaccgcccctcaggtatcgcggtcaccccggatggattgatcgtcgtggtggactttggcaacaatcgaatcctcatcttctaattgtgtcttctggggttttctgtgtttagggtgtgcatgtgtgtgtctctcatttttgaatttcaaagaagaaatcgtctcagggatatttctttttctttttccccccctttatttttattatttttttttttttaagaacaaaagtacaacattgcctaagtcctacctcagcttaaatttttttttttttttttttttttacagatgaatgtaattctcctctgtgcagggcttgagcctgtgaagtgataatttctatctacctcaactctttgcatttccctctctggcaagcctcttggctcacatcctcttccctcctgaaggcctaggaggcacaggacagcggtcctgcaaccttgagggcactggaagcatatggtggggtgcatgcattttgtagattgagccaaggaaacccaaaaactactaagtaaaaaagagaagtataagatgttggaaagataggatttaaaattcataattgtagtgtatttgtgttcttgagaatactgtgtttatgtggggttaggttgtgtgtggtttgctcttttcttttttatcgatgcaacagggcttttctctgtttacctcagtgtgtctgacatctcatccctaggagatatggctgtggcttgctagcctagaaaagcagaccattcatattttcgggtgtctcatctttgtatctggtaaagaagggtgggtgtgtgggagtgtgtgtgtgcacacgcgtgcatgtttgtgtttcaaagccatctttgcttgaggagaaaagctggacttgacacaagacatcccaagcaaatttcttcccagtttgctaagaaactcgatttagctcaggactctaggttgaaagtgttcgcaaaggttacgtgtctgtgtttttgttgtttgttataaagacttaggtataagaaaataagttaaccttgttctcagagagagaggggcttttcagatgtggaccagggtgccacagaagtggggggtgctggagaagtgaggcacccccacatcagggactgttcaggagggcgggggacctcgtctctatgtgtgagggtgtggtaggcaaagggatagctttgatttgagaagcacaatgttttttcctgctttggaagttcagggaatgtagaagattgtctgactggtgggagcacctcactccctaacccttggctcaggtctgtttctcaggaaccttccctaagtctgagtggaggcagacctcaaggttaacacaaagccccgttcctcccccaaaggcctttgaatgtaaaggatttacaaaccaaaccacccaacaggaagtgtaggacacagtatatgtctttttttttttttaattttatttt

SEQ ID NO: 5 - protein, 832 aa, Drosophila melanogaster
MMELLSNNSVPQQMASSNAPSANNVAHSSTANGSGGGSVSSNASNSSERLLAGILESFPAWDLNVGLLPNVGQSSPPRADFFINNFLGGLDTHGDFSIGPIGSGARSNPKMSPESSNNSSISCGWCEVSASIRCLECNEFMCNDCLREHRNSPLSSNHSIVSLPTPIGASPTGGSSVNAQTPPSGNFICDIHNEMLRYVCDYCRKLVCQCCTLHEHKEHSYASIQSFMVGSKEKLEGAIESSQVGTRCIKSSIDKALAFIRLIERNCSELSDNIRKAFRQFIIAIEDRERFLLDFVEKLRQRRLAILHDQMAGLKSALAGLSETSDMLSKVADNACNMDQIEIAMKLTNGQRQMEQFAGIYKDLQPKQEVFAFAPPDYSLLQDIRNQGGVILVDDKNLPIVSSSNGIVPSVSSVNAVAAASVGVVGGVAGVVGGVGVSNGLDLAFGMNMPNNPLSVASSSVRRPLLRDNSFRIPSPIMQPRGGSACGMSSGMSSAALDWELNGLRSSPGLHFSAPRTTQAIPGCMDLVKVRNSNALSLSFATEGHEDGQVSRPWGLCVDKMGHVLVSDRRNNRVQVFNPDGSLKFKFGRKGVGNGEFDLPAGICVDVDNRIIVVDKDNHRVQIFTASGVFLLKFGSYGKEYGQFQYPWDVAVNSRRQIVVTDSRNHRIQQFDSEGRFIRQIVFDNHGQTKGIASPRGVCYTPTGNIIVSDFDNHCLYLIDPDINDILSVKGHEGSGFHEFNRPSGLCCDDEGRIIVADSKNQRILVFNQNLDFMWDIEVRPSINPLMPPTLDEKDRTCDVAIMPDGRIVFLIELSPDSKEGSNPYKRFVHVF

SEQ ID NO: 6 - protein, 832 aa, Drosophila melanogaster; identical to SEQ ID NO: 5

SEQ ID NO: 7 - protein, 832 aa, Drosophila melanogaster; identical to SEQ ID NO: 5

SEQ ID NO: 8 - DNA, 4516 bp, Drosophila melanogaster
cagcggcgagcgcatgaacgtgagttttgcagtttgatggttaaatgcgtgcgtgaaggttggagtgaagattttgagccgaattcgaatcctagtccccgccgcaataagtgggtcagaattggagccgttcgattgggccaaacaaaaagctgggcgccaccgcagactgcagctaaaacacgccgccaaaaaggaaaagtgcacgagggtggaaccaagggtaaataagattcgcccgaaaacgcttggaaaatgtaaacaatctgtaagcacagcgacgataagcgattttcaaagagtttgccagctttttacatcgatcaaacactcgcataataaacgaataagaactgacgattaaatcgaattgaagtggagtgacgactatgggataccctaggaccgcgtagagtgaatgaatttgacaacggaaaagcgccactccattgactgctgccttgtgctatagaaacgtgatctcaccactcttatgatggaacttttgtcaaacaattcggttccgcaacaaatggccagcagcaatgcgccgagcgccaacaatgtggcccactcctcaacggccaacggaagtggaggcggcagtgtgagcagcaatgccagcaactcctccgagcgactgctggcgggcatcctggagagctttcccgcctgggacctcaacgtgggactactgcccaacgtgggccagagctcaccaccacgcgcagacttctttatcaataacttcctgggcggcctggacacgcatggcgacttcagcattggacccattggcagcggtgcccgcagcaatccgaagatgtcgccagagtcatctaacaactcgagcatcagctgcggttggtgcgaggttagtgcttcaatacgctgcctggagtgcaacgagttcatgtgcaacgactgcctaagggagcatcgcaacagtccgctgtcctccaaccactcgatcgtgtccttgcccacgcccattggagcctcgcccacgggtggcagctcggtaaatgcacagactccgcccagcggcaactttatctgcgacatacacaacgagatgttgcgctacgtatgtgactactgccggaaattggtgtgtcagtgctgcacactgcacgagcacaaggagcacagctacgcgtccatccaaagctttatggtgggctcgaaggagaagctggagggcgccattgagagcagccaggtgggcacgcgctgcattaagagcagcattgacaaagcgttggccttcatccggcttatcgagcgcaattgcagcgagctgagcgataatatacgcaaggcattccgtcagtttatcattgccatcgaggaccgcgagcgtttcctcctggactttgtggaaaagctccgccagcgtcgtctggccatcctacacgatcagatggcaggcttaaagtctgctctcgccggactctccgaaacgtccgatatgcttagcaaggtggcggacaatgcctgcaacatggaccagattgaaattgccatgaagttgaccaatgggcagaggcagatggagcagtttgcgggcatatataaggacctgcagccaaaacaggaagtctttgccttcgcaccaccagattacagcctgctacaggatatccgcaaccagggtggcgttatcctggtggacgacaagaacttgcccatcgtctctagcagcaacggaattgtgccgagcgtctccagtgttaatgccgtggccgcggcctccgtgggagttgtgggaggcgttgctggagtagtcggaggcgttggcgtgtcaaacggcctggacttggccttcggcatgaacatgcccaacaatccgctgagtgttgcttcctcaagtgtgcgacgtcccctgttgcgagacaatagcttccggattccatcgcccataatgcaaccacgcggaggaagtgcctgtgggatgtccagtgggatgtccagcgctgctcttgattgggaactcaatggactgcgcagctcgccaggattacacttcagtgcgccgcgaaccacgcaggccattccgggatgcatggatctggtcaaggtgcgtaactcgaatgctctctcgctatccttcgccaccgagggccacgaggacggacaggtgagccggccatggggtctatgcgttgataaaatgggccacgttctcgtatcggatcgccgcaacaatcgcgtccaggtcttcaatcccgatggctccctaaagttcaaattcggccgtaaaggcgtgggcaatggagagttcgatctgcctgctggaatctgtgttgacgttgataatcgcattattgtggtcgacaaggacaatcaccgtgtgcaaatctttaccgccagcggcgtcttcctgctcaagttcggcagctacggcaaggagtacggccagttccagtatccgtgggatgtggccgtgaactcgcgccgccaaattgtggttaccgactcgcgcaatcatcgcatccagcagttcgactctgagggtcgcttcatccgccagatagtgttcgacaaccatgggcaaaccaaaggaatcgcctcgccacgaggcgtttgctacacgccgacgggcaacatcatagtgtctgactttgacaaccactgcctctacctgattgatccagacatcaacgacattctttccgtgaagggacatgagggctcgggcttccatgaatttaatcgaccctccggcctgtgctgtgacgacgagggccgcattatagtggccgattcaaagaatcaacgcatcctggtcttcaatcagaacctggactttatgtgggatatcgaggtgcgaccctccatcaatcccttgatgccgcccacgctggacgagaaagatcgcacttgcgatgtggccataatgccagacggtcgcattgtgttcctcattgaactgtcgccagactccaaggaagggtcaaacccttacaagcggtttgtgcacgtattctaaataaggccacgagtgccgatctttttttttagaccatttacaagagcgaaaccttagcgtagagtttcaaataacatgacgattatgaggaggtgccgaaaccgatttaataagaaaaaatctgaaatcatgaaagttagcgaaaatttttagcttaagtgctacaacgattacctcgatattgcttaagagttatggttacgatttggtaatttacaatacaatacgaattacacatatgtatctatggatatctatatctatacgcatatgcatatatctagcgaatatacaagcccaaccaactagaactataactaaaacaaaaacaaaaaacaaaaactaaagcgaaaggcaggcggttgagtaggatttaggagatatatataaacgtagggtccgtgagcattcagtagagttaccgaacctagtaaaagataatgcaaagagggaactcaaccatttactagatgaaaatattattacctgttagttagactgaaaggcagtttgctagctgatagaaggaggtacgaaccaggtcaggtggcgattgtgtttttttttacgggtttgcttagcggaatgaggaaactacattcgacaagagttactaaacatgatttgcagaaacactgtatggtatggtattaatgtttgtattatatttatagttcgtttgttcagttctgtttttcgtttattttaaaattactttttttttgggacatacatatgtattttaatgatttccttataattgagactgagcatttgacaacacgaactttgcgtgtacggaagcaaagcgaaacaaaacaaagaaacaaacaaaaaacaagaaaatattgatggaacccgctgacttttcgttgaactttatacaaagactgattaactaaaccgcaatttcggagccaactaaagaaccatatatggtgaggccaaaataagtgaaagagcaacacactcaaattgcaatgacattgataacgtaaagtaacttaacatagcttaacatacatactttcgcgcatatagagcgatacagatatcatatagatccgatcatccagtcacgaatcatcatgggcatagctgaaaccgagtttaactgacattcatcgacatatttacccatccatccatccatccatccactcactcataccgatcatgcatcacagggctttgtttttcgtttgtcgttactttgtaattattgtgaaaggatttagccacaggatacagattcacggcaggttgaaaggaggaggaggaggagggaggggttagctaaaattagtgggacattttgggaagtaaacgaaacagacctgaaacaaaataacgaagaagttataatttttgactcaatgtgttcattagcatttagtagacttagcgggcgtggcatagtgaaaacgtatggaaaatccagagcaatataagggcgtattgaatacacatttaggtgacatgttcctcgcaaaagaaacaaataaaacaaaacaaaagaaaaca

SEQ ID NO: 9 - DNA, 4591 bp, Drosophila melanogaster
cagcggcgagcgcatgaacgtgagttttgcagtttgatggttaaatgcgtgcgtgaaggttggagtgaagattttgagccgaattcgaatcctagtccccgccgcaataagtgggtcagaattggagccgttcgattgggccaaacaaaaagctgggcgccaccgcagactgcagctaaaacacgccgccaaagtaattggaagaagaagcaggcgcacaataggaataattgttcatggattttttcgatttctcgttcgcttgcagaaggaaaagtgcacgagggtggaaccaagggtaaataagattcgcccgaaaacgcttggaaaatgtaaacaatctgtaagcacagcgacgataagcgattttcaaagagtttgccagctttttacatcgatcaaacactcgcataataaacgaataagaactgacgattaaatcgaattgaagtggagtgacgactatgggataccctaggaccgcgtagagtgaatgaatttgacaacggaaaagcgccactccattgactgctgccttgtgctatagaaacgtgatctcaccactcttatgatggaacttttgtcaaacaattcggttccgcaacaaatggccagcagcaatgcgccgagcgccaacaatgtggcccactcctcaacggccaacggaagtggaggcggcagtgtgagcagcaatgccagcaactcctccgagcgactgctggcgggcatcctggagagctttcccgcctgggacctcaacgtgggactactgcccaacgtgggccagagctcaccaccacgcgcagacttctttatcaataacttcctgggcggcctggacacgcatggcgacttcagcattggacccattggcagcggtgcccgcagcaatccgaagatgtcgccagagtcatctaacaactcgagcatcagctgcggttggtgcgaggttagtgcttcaatacgctgcctggagtgcaacgagttcatgtgcaacgactgcctaagggagcatcgcaacagtccgctgtcctccaaccactcgatcgtgtccttgcccacgcccattggagcctcgcccacgggtggcagctcggtaaatgcacagactccgcccagcggcaactttatctgcgacatacacaacgagatgttgcgctacgtatgtgactactgccggaaattggtgtgtcagtgctgcacactgcacgagcacaaggagcacagctacgcgtccatccaaagctttatggtgggctcgaaggagaagctggagggcgccattgagagcagccaggtgggcacgcgctgcattaagagcagcattgacaaagcgttggccttcatccggcttatcgagcgcaattgcagcgagctgagcgataatatacgcaaggcattccgtcagtttatcattgccatcgaggaccgcgagcgtttcctcctggactttgtggaaaagctccgccagcgtcgtctggccatcctacacgatcagatggcaggcttaaagtctgctctcgccggactctccgaaacgtccgatatgcttagcaaggtggcggacaatgcctgcaacatggaccagattgaaattgccatgaagttgaccaatgggcagaggcagatggagcagtttgcgggcatatataaggacctgcagccaaaacaggaagtctttgccttcgcaccaccagattacagcctgctacaggatatccgcaaccagggtggcgttatcctggtggacgacaagaacttgcccatcgtctctagcagcaacggaattgtgccgagcgtctccagtgttaatgccgtggccgcggcctccgtgggagttgtgggaggcgttgctggagtagtcggaggcgttggcgtgtcaaacggcctggacttggccttcggcatgaacatgcccaacaatccgctgagtgttgcttcctcaagtgtgcgacgtcccctgttgcgagacaatagcttccggattccatcgcccataatgcaaccacgcggaggaagtgcctgtgggatgtccagtgggatgtccagcgctgctcttgattgggaactcaatggactgcgcagctcgccaggattacacttcagtgcgccgcgaaccacgcaggccattccgggatgcatggatctggtcaaggtgcgtaactcgaatgctctctcgctatccttcgccaccgagggccacgaggacggacaggtgagccggccatggggtctatgcgttgataaaatgggccacgttctcgtatcggatcgccgcaacaatcgcgtccaggtcttcaatcccgatggctccctaaagttcaaattcggccgtaaaggcgtgggcaatggagagttcgatctgcctgctggaatctgtgttgacgttgataatcgcattattgtggtcgacaaggacaatcaccgtgtgcaaatctttaccgccagcggcgtcttcctgctcaagttcggcagctacggcaaggagtacggccagttccagtatccgtgggatgtggccgtgaactcgcgccgccaaattgtggttaccgactcgcgcaatcatcgcatccagcagttcgactctgagggtcgcttcatccgccagatagtgttcgacaaccatgggcaaaccaaaggaatcgcctcgccacgaggcgtttgctacacgccgacgggcaacatcatagtgtctgactttgacaaccactgcctctacctgattgatccagacatcaacgacattctttccgtgaagggacatgagggctcgggcttccatgaatttaatcgaccctccggcctgtgctgtgacgacgagggccgcattatagtggccgattcaaagaatcaacgcatcctggtcttcaatcagaacctggactttatgtgggatatcgaggtgcgaccctccatcaatcccttgatgccgcccacgctggacgagaaagatcgcacttgcgatgtggccataatgccagacggtcgcattgtgttcctcattgaactgtcgccagactccaaggaagggtcaaacccttacaagcggtttgtgcacgtattctaaataaggccacgagtgccgatctttttttttagaccatttacaagagcgaaaccttagcgtagagtttcaaataacatgacgattatgaggaggtgccgaaaccgatttaataagaaaaaatctgaaatcatgaaagttagcgaaaatttttagcttaagtgctacaacgattacctcgatattgcttaagagttatggttacgatttggtaatttacaatacaatacgaattacacatatgtatctatggatatctatatctatacgcatatgcatatatctagcgaatatacaagcccaaccaactagaactataactaaaacaaaaacaaaaaacaaaaactaaagcgaaaggcaggcggttgagtaggatttaggagatatatataaacgtagggtccgtgagcattcagtagagttaccgaacctagtaaaagataatgcaaagagggaactcaaccatttactagatgaaaatattattacctgttagttagactgaaaggcagtttgctagctgatagaaggaggtacgaaccaggtcaggtggcgattgtgtttttttttacgggtttgcttagcggaatgaggaaactacattcgacaagagttactaaacatgatttgcagaaacactgtatggtatggtattaatgtttgtattatatttatagttcgtttgttcagttctgtttttcgtttattttaaaattactttttttttgggacatacatatgtattttaatgatttccttataattgagactgagcatttgacaacacgaactttgcgtgtacggaagcaaagcgaaacaaaacaaagaaacaaacaaaaaacaagaaaatattgatggaacccgctgacttttcgttgaactttatacaaagactgattaactaaaccgcaatttcggagccaactaaagaaccatatatggtgaggccaaaataagtgaaagagcaacacactcaaattgcaatgacattgataacgtaaagtaacttaacatagcttaacatacatactttcgcgcatatagagcgatacagatatcatatagatccgatcatccagtcacgaatcatcatgggcatagctgaaaccgagtttaactgacattcatcgacatatttacccatccatccatccatccatccactcactcataccgatcatgcatcacagggctttgtttttcgtttgtcgttactttgtaattattgtgaaaggatttagccacaggatacagattcacggcaggttgaaaggaggaggaggaggagggaggggttagctaaaattagtgggacattttgggaagtaaacgaaacagacctgaaacaaaataacgaagaagttataatttttgactcaatgtgttcattagcatttagtagacttagcgggcgtggcatagtgaaaacgtatggaaaatccagagcaatataagggcgtattgaatacacatttaggtgacatgttcctcgcaaaagaaacaaataaaacaaaacaaaagaaaaca

SEQ ID NO: 10 - DNA, 4367 bp, Drosophila melanogaster
agagaagaagaggaagaggcaacggcagcggcgagcgcatgaacaaggaaaagtgcacgagggtggaaccaagggtaaataagattcgcccgaaaacgcttggaaaatgtaaacaatctgtaagcacagcgacgataagcgattttcaaagagtttgccagctttttacatcgatcaaacactcgcataataaacgaataagaactgacgattaaatcgaattgaagtggagtgacgactatgggataccctaggaccgcgtagagtgaatgaatttgacaacggaaaagcgccactccattgactgctgccttgtgctatagaaacgtgatctcaccactcttatgatggaacttttgtcaaacaattcggttccgcaacaaatggccagcagcaatgcgccgagcgccaacaatgtggcccactcctcaacggccaacggaagtggaggcggcagtgtgagcagcaatgccagcaactcctccgagcgactgctggcgggcatcctggagagctttcccgcctgggacctcaacgtgggactactgcccaacgtgggccagagctcaccaccacgcgcagacttctttatcaataacttcctgggcggcctggacacgcatggcgacttcagcattggacccattggcagcggtgcccgcagcaatccgaagatgtcgccagagtcatctaacaactcgagcatcagctgcggttggtgcgaggttagtgcttcaatacgctgcctggagtgcaacgagttcatgtgcaacgactgcctaagggagcatcgcaacagtccgctgtcctccaaccactcgatcgtgtccttgcccacgcccattggagcctcgcccacgggtggcagctcggtaaatgcacagactccgcccagcggcaactttatctgcgacatacacaacgagatgttgcgctacgtatgtgactactgccggaaattggtgtgtcagtgctgcacactgcacgagcacaaggagcacagctacgcgtccatccaaagctttatggtgggctcgaaggagaagctggagggcgccattgagagcagccaggtgggcacgcgctgcattaagagcagcattgacaaagcgttggccttcatccggcttatcgagcgcaattgcagcgagctgagcgataatatacgcaaggcattccgtcagtttatcattgccatcgaggaccgcgagcgtttcctcctggactttgtggaaaagctccgccagcgtcgtctggccatcctacacgatcagatggcaggcttaaagtctgctctcgccggactctccgaaacgtccgatatgcttagcaaggtggcggacaatgcctgcaacatggaccagattgaaattgccatgaagttgaccaatgggcagaggcagatggagcagtttgcgggcatatataaggacctgcagccaaaacaggaagtctttgccttcgcaccaccagattacagcctgctacaggatatccgcaaccagggtggcgttatcctggtggacgacaagaacttgcccatcgtctctagcagcaacggaattgtgccgagcgtctccagtgttaatgccgtggccgcggcctccgtgggagttgtgggaggcgttgctggagtagtcggaggcgttggcgtgtcaaacggcctggacttggccttcggcatgaacatgcccaacaatccgctgagtgttgcttcctcaagtgtgcgacgtcccctgttgcgagacaatagcttccggattccatcgcccataatgcaaccacgcggaggaagtgcctgtgggatgtccagtgggatgtccagcgctgctcttgattgggaactcaatggactgcgcagctcgccaggattacacttcagtgcgccgcgaaccacgcaggccattccgggatgcatggatctggtcaaggtgcgtaactcgaatgctctctcgctatccttcgccaccgagggccacgaggacggacaggtgagccggccatggggtctatgcgttgataaaatgggccacgttctcgtatcggatcgccgcaacaatcgcgtccaggtcttcaatcccgatggctccctaaagttcaaattcggccgtaaaggcgtgggcaatggagagttcgatctgcctgctggaatctgtgttgacgttgataatcgcattattgtggtcgacaaggacaatcaccgtgtgcaaatctttaccgccagcggcgtcttcctgctcaagttcggcagctacggcaaggagtacggccagttccagtatccgtgggatgtggccgtgaactcgcgccgccaaattgtggttaccgactcgcgcaatcatcgcatccagcagttcgactctgagggtcgcttcatccgccagatagtgttcgacaaccatgggcaaaccaaaggaatcgcctcgccacgaggcgtttgctacacgccgacgggcaacatcatagtgtctgactttgacaaccactgcctctacctgattgatccagacatcaacgacattctttccgtgaagggacatgagggctcgggcttccatgaatttaatcgaccctccggcctgtgctgtgacgacgagggccgcattatagtggccgattcaaagaatcaacgcatcctggtcttcaatcagaacctggactttatgtgggatatcgaggtgcgaccctccatcaatcccttgatgccgcccacgctggacgagaaagatcgcacttgcgatgtggccataatgccagacggtcgcattgtgttcctcattgaactgtcgccagactccaaggaagggtcaaacccttacaagcggtttgtgcacgtattctaaataaggccacgagtgccgatctttttttttagaccatttacaagagcgaaaccttagcgtagagtttcaaataacatgacgattatgaggaggtgccgaaaccgatttaataagaaaaaatctgaaatcatgaaagttagcgaaaatttttagcttaagtgctacaacgattacctcgatattgcttaagagttatggttacgatttggtaatttacaatacaatacgaattacacatatgtatctatggatatctatatctatacgcatatgcatatatctagcgaatatacaagcccaaccaactagaactataactaaaacaaaaacaaaaaacaaaaactaaagcgaaaggcaggcggttgagtaggatttaggagatatatataaacgtagggtccgtgagcattcagtagagttaccgaacctagtaaaagataatgcaaagagggaactcaaccatttactagatgaaaatattattacctgttagttagactgaaaggcagtttgctagctgatagaaggaggtacgaaccaggtcaggtggcgattgtgtttttttttacgggtttgcttagcggaatgaggaaactacattcgacaagagttactaaacatgatttgcagaaacactgtatggtatggtattaatgtttgtattatatttatagttcgtttgttcagttctgtttttcgtttattttaaaattactttttttttgggacatacatatgtattttaatgatttccttataattgagactgagcatttgacaacacgaactttgcgtgtacggaagcaaagcgaaacaaaacaaagaaacaaacaaaaaacaagaaaatattgatggaacccgctgacttttcgttgaactttatacaaagactgattaactaaaccgcaatttcggagccaactaaagaaccatatatggtgaggccaaaataagtgaaagagcaacacactcaaattgcaatgacattgataacgtaaagtaacttaacatagcttaacatacatactttcgcgcatatagagcgatacagatatcatatagatccgatcatccagtcacgaatcatcatgggcatagctgaaaccgagtttaactgacattcatcgacatatttacccatccatccatccatccatccactcactcataccgatcatgcatcacagggctttgtttttcgtttgtcgttactttgtaattattgtgaaaggatttagccacaggatacagattcacggcaggttgaaaggaggaggaggaggagggaggggttagctaaaattagtgggacattttgggaagtaaacgaaacagacctgaaacaaaataacgaagaagttataatttttgactcaatgtgttcattagcatttagtagacttagcgggcgtggcatagtgaaaacgtatggaaaatccagagcaatataagggcgtattgaatacacatttaggtgacatgttcctcgcaaaagaaacaaataaaacaaaacaaaagaaaaca

SEQ ID NO: 11 - protein, 263 aa, human
LCRPWGVSVDKEGYIIVADRSNNRIQVFKPCGAFHHKFGTLGSRPGQFDRPAGVACDASRRIVVADKDNHRIQIFTFEGQFLLKFGEKGTKNGQFNYPWDVAVNSEGKILVSDTRNHRIQLFGPDGVFLNKYGFEGALWKHFDSPRGVAFNHEGHLVVTDFNNHRLLVIHPDCQSARFLGSEGTGNGQFLRPQGVAVDQEGRIIVADSRNHRVQMFESNGSFLCKFGAQGSGFGQMDRPSGIAITPDGMIVVVDFGNNRILVF

SEQ ID NO: 12 - DNA, 792 bp, human
ctctgccgcccttggggtgtgagtgtagacaaggagggctacatcattgtcgccgaccgcagcaacaaccgcatccaggtgttcaagccctgcggcgccttccaccacaaattcggcaccctgggctcccggcctgggcagttcgaccgaccagccggcgtggcctgtgacgcctcacgcaggatcgtggtggctgacaaggacaatcatcgcatccagatcttcacgttcgagggccagttcctcctcaagtttggtgagaaaggaaccaagaatgggcagttcaactacccttgggatgtggcggtgaattctgagggcaagatcctggtctcagacacgaggaaccaccggatccagctgtttgggcctgatggtgtcttcctaaacaagtatggcttcgagggggctctctggaagcactttgactccccacggggtgtggccttcaaccatgagggccacttggtggtcactgacttcaacaaccaccggctcctggttattcaccccgactgccagtcggcacgctttctgggctcggagggcacaggcaatgggcagttcctgcgcccacaaggggtagctgtggaccaggaagggcgcatcattgtggcggattccaggaaccatcgggtacagatgtttgaatccaacggcagcttcctgtgcaagtttggtgctcaaggcagcggctttgggcagatggaccgcccttccggcatcgccatcacccccgacggaatgatcgttgtggtggactttggcaacaatcgaatcctcgtcttctaa

SEQ ID NO: 13 - protein, 263 aa, Mus musculus
LCRPWGVSVDKEGFIIVADRSNNRIQVFKPCGSFHHKFGTLGSRPGQFDRPAGVACDASRRIIVADKDNHRIQIFTFEGQFLLKFGEKGTKNGQFNYPWDVAVNSEGKILVSDTRNHRIQLFGPDGVFLNKYGFEGSLWKHFDSPRGVAFNNEGHLVVTDFNNHRLLVIHPDCQSARFLGSEGSGNGQFLRPQGVAVDQEGRIIVADSRNHRVQMFEANGSFLCKFGAQGSGFGQMDRPSGIAVTPDGLIVVVDFGNNRILIF

SEQ ID NO: 14 - DNA, 852 bp, Mus musculus
tacgtgggcatcgggctccccgggctgagcttcggcagtgaaggtgacggggagggcaagctctgcaggccctggggcgtgagtgttgataaggagggtttcatcatagtggctgaccgaagcaataacagaatccaggtgtttaagccctgtggttccttccaccacaagtttggtacccttgggtctcgtcctgggcagtttgaccgaccggctggggtggcttgtgacgcctcccgcaggatcatagtggccgacaaagacaatcatcgcattcagatcttcaccttcgagggccagttccttcttaagtttggtgagaaaggcaccaagaacgggcagtttaactacccgtgggatgtagcagtgaattctgagggcaagatcctcgtttcagacacccggaatcatcgcatccagttgttcggaccggacggggtcttcctgaacaagtacggcttcgagggatctctctggaagcacttcgactctccacggggcgtggctttcaacaatgaaggtcacctggtagtcaccgacttcaacaaccaccggcttctggttattcaccctgattgccaatctgcccgcttcttgggttcggagggctcgggcaatgggcagttcctgcgaccacagggtgtggctgtggaccaggaagggagaatcatcgtagccgattccagaaatcaccgcgtgcagatgtttgaggccaatggcagcttcctgtgcaagtttggtgctcaagggagtggcttcgggcagatggaccgcccctcaggtatcgcggtcaccccggatggattgatcgtcgtggtggactttggcaacaatcgaatcctcatcttctaa

SEQ ID NO: 15 - protein, 283 aa, Drosophila melanogaster
VSRPWGLCVDKMGHVLVSDRRNNRVQVFNPDGSLKFKFGRKGVGNGEFDLPAGICVDVDNRIIVVDKDNHRVQIFTASGVFLLKFGSYGKEYGQFQYPWDVAVNSRRQIVVTDSRNHRIQQFDSEGRFIRQIVFDNHGQTKGIASPRGVCYTPTGNIIVSDFDNHCLYLIDPDINDILSVKGHEGSGFHEFNRPSGLCCDDEGRIIVADSKNQRILVFNQNLDFMWDIEVRPSINPLMPPTLDEKDRTCDVAIMPDGRIVFLIELSPDSKEGSNPYKRFVHVF

SEQ ID NO: 16 - DNA, 1293 bp, Drosophila melanogaster
gtgagccggccatggggtctatgcgttgataaaatgggccacgttctcgtatcggatcgccgcaacaatcgcgtccaggtcttcaatcccgatggctccctaaagttcaaattcggccgtaaaggcgtgggcaatggagagttcgatctgcctgctggaatctgtgttgacgttgataatcgcattattgtggtcgacaaggacaatcaccgtgtgcaaatctttaccgccagcggcgtcttcctgctcaagttcggcagctacggcaaggagtacggccagttccagtatccgtgggatgtggccgtgaactcgcgccgccaaattgtggttaccgactcgcgcaatcatcgcatccagcagttcgactctgagggtcgcttcatccgccagatagtgttcgacaaccatgggcaaaccaaaggaatcgcctcgccacgaggcgtttgctacacgccgacgggcaacatcatagtgtctgactttgacaaccactgcctctacctgattgatccagacatcaacgacattctttccgtgaagggacatgagggctcgggcttccatgaatttaatcgaccctccggcctgtgctgtgacgacgagggccgcattatagtggccgattcaaagaatcaacgcatcctggtcttcaatcagaacctggactttatgtgggatatcgaggtgcgaccctccatcaatcccttgatgccgcccacgctggacgagaaagatcgcacttgcgatgtggccataatgccagacggtcgcattgtgttcctcattgaactgtcgccagactccaaggaagggtcaaacccttacaagcggtttgtgcacgtattctaaataaggccacgagtgccgatctttttttttagaccatttacaagagcgaaaccttagcgtagagtttcaaataacatgacgattatgaggaggtgccgaaaccgatttaataagaaaaaatctgaaatcatgaaagttagcgaaaatttttagcttaagtgctacaacgattacctcgatattgcttaagagttatggttacgatttggtaatttacaatacaatacgaattacacatatgtatctatggatatctatatctatacgcatatgcatatatctagcgaatatacaagcccaaccaactagaactataactaaaacaaaaacaaaaaacaaaaactaaagcgaaaggcaggcggttgagtaggatttaggagatatatataaacgtagggtccgtgagcattcagtagagttaccgaacctagtaaaagataa

SEQ ID NO: 17 - protein, 16 aa, Mus musculus
KFGEKGTKNGQFNYPW

SEQ ID NO: 18 - protein, 16 aa, Mus musculus
CVRAHQRVRLTKDHYI

SEQ ID NO: 19 - protein, 15 aa, Drosophila melanogaster
LSLSFATEGHEDGQV

SEQ ID NO: 20 - protein, 18 aa, Drosophila melanogaster
SPDSKEGSNPYKRFVHVF

SEQ ID NO: 21 - DNA, 20 bp, Homo sapiens
ccgtgtgcgaccagaaagta

SEQ ID NO: 22 - DNA, 21 bp, Homo sapiens
ccagatctgcttgctgtgcaa

SEQ ID NO: 23 - DNA, 21 bp, Homo sapiens
tgggacatacgtggtgagtta

SEQ ID NO: 24 - DNA, 786 bp, Homo sapiens
atcgcaacagtccgctgtcctccaaccactcgatcgtgtccttgcccacgcccattggagcctcgcccacgggtggcagctcggtaaatgcacagactccgcccagcggcaactttatctgcgacatacacaacgagatgttgcgctacgtatgtgactactgccggaaattggtgtgtcagtgctgcacactgcacgagcacaaggagcacagctacgcgtccatccaaagctttatggtgggctcgaaggagaagctggagggcgccattgagagcagccaggtgggcacgcgctgcattaagagcagcattgacaaagcgttggccttcatccggcttatcgagcgcaattgcagcgagctgagcgataatatacgcaaggcattccgtcagtttatcattgccatcgaggaccgcgagcgtttcctcctggactttgtggaaaagctccgccagcgtcgtctggccatcctacacgatcagatggcaggcttaaagtctgctctcgccggactctccgaaacgtccgatatgcttagcaaggtggcggacaatgcctgcaacatggaccagattgaaattgccatgaagttgaccaatgggcagaggcagatggagcagtttgcgggcatatataaggacctgcagccaaaacaggaagtctttgccttcgcaccaccagattacagcctgctacaggatatccgcaaccagggtggcgttatcctggtggacgacaagaacttgcccatcgtctctagcagcaacggaattgtgccgagcg

(19) United States
(12) Patent Application Publication
(10) Pub. No.: US 2011/0224279 A1
(43) Pub. Date: Sep. 15, 2011

(54) ANTICANCER AGENT

(75) Inventors: Waldemar Kolanus, Bonn (DE); Michael Karl Hoch, Swisttal (DE); Joachim L. Schultze, Konigswinter (DE); Birgit Loeer, Sankt Augustin (DE)

(73) Assignee: RHEINISCHE FRIEDRICH-WILHELMS UNIVERSITAT, Bonn (DE)

(21) Appl. No.: 12/920,167

(22) PCT Filed: Feb. 27, 2009
(86) PCT No.: PCT/EP2009/052389

§ 371 Date: Aug. 30, 2010

(30) Foreign Application Priority Data
Feb. 29, 2008 (EP) 08152180.9

(51) Int. Cl. A61K 31/713; C07H 21/02; C07K 7/08; C12N 15/12; C07K 16/00; C07K 9/00; C12Q 1/00; C12Q 1/68; A61P 35/00

(52) U.S. Cl. 514/44 A; 536/24.5; 530/326; 536/23.5; 530/387.3; 530/322; 435/40.5; 435/6.14

(57) ABSTRACT

The present invention relates to a nucleic acid molecule and a pharmaceutical or diagnostic composition for the therapeutic and/or prophylactic treatment or diagnosis of cancer and/or metastasis thereof, comprising a nucleic acid molecule, or an amino acid sequence related to Trim71 and/or its mammalian and non mammalian orthologs and/or a nucleic acid sequence of the gene encoding for Trim71 and/or its mammalian and non mammalian orthologs.

The present invention relates to a pharmaceutical or diagnostic composition. More particularly, it concerns the use of new genes and their respective encoded proteins in a pharmaceutical or diagnostic composition for the treatment of cancer. Further, the present invention relates to nucleic acid molecules and the use of nucleic acid molecules for preparing a medicament for therapeutic or prophylactic treatment and/or diagnosis of cancer.

Cancer is a major world-wide health problem. Although extensive research around the world has led to advances in cancer treatment, progress has been slow and there is no known cure. The control of cancer is still a most important subject on today's medicine, and new cancer therapy and new anti-cancer agents are topics of utmost interest among medical and pharmaceutical researchers.

Known methods of cancer treatment for example use chemotherapeutic anti-cancer agents. However, the use of existing compounds such as alkylating agents making use of cytotoxicity is considerably limited owing to manifest side effects. Moreover, tumor cell resistance to chemotherapeutic agents represents a significant problem in clinical oncology, being one of the main reasons why many of the most prevalent forms of human cancer still resist effective chemotherapeutic intervention, despite certain advances in the field of chemotherapy.

Other known methods in cancer treatment use antibodies. However, antibodies are not free of serious side effects either. Moreover, antigen-negative or antigen-deficient cells can survive and repopulate the tumor or lead to further metastases. Moreover, antibodies are not a treatment of choice for treating solid tumors.

However, modern molecular biological techniques have contributed to our understanding of the genetic aspects of cancer development. So, numerous scientific studies have established that in the development of cancer gene mutations are involved including inactivation of tumor suppressor genes. Tumor-suppressor genes are genes that normally prevent cells from growing out of control. The loss or silencing of one or more tumor-suppressor genes is believed to be an important part of cancer development. Therefore, the administration of tumor suppressor genes is another useful strategy for the prevention and/or treatment of cancer. Another type of genes involved in the development of cancer are oncogenes, which promote cell growth or cell division.

However, new therapeutic modalities for the treatment of cancer are urgently needed.

Therefore, an object of the present invention is to provide a novel agent for the treatment or diagnosis of cancer.

This object is met with means according to the independent claims of the present invention. The dependent claims are related to preferred embodiments.

The term “cancer” as used herein refers to or describes the physiological condition, preferably in a mammalian subject, that is typically characterized by abnormal or unregulated cell growth, often being capable of invading adjacent tissues and spreading to distant organs. The term “cancer” as used herein includes carcinomas, germ cell tumors, neoplasms particularly malignant neoplasms or malignant tumors, and pre-malignant conditions.

Cancer is usually classified according to the tissue from which the cancerous cells originate, as well as the normal cell type they most resemble. Examples of cancer include, but are not limited to, the group comprising thyroid cancer, lung cancer, small cell lung cancer (SCLC), liver cancer, cancers of the kidney, cancers of the atrioventricular node, cancers of the skeletal muscle, skin cancer, salivary gland cancer, ovary cancer, upper gastrointestinal cancers preferably selected from the group comprising pancreas cancer, esophagus cancer, and/or stomach cancer, and/or cancers of the nervous system preferably selected from the group comprising cancers of the cingulated cortex, the Medulla oblongata, Temporal lobe, Ciliary ganglion, and/or the Superior cervical ganglion.

The term “carcinoma” refers to the tissue resulting from abnormal or unregulated cell growth.

As used herein, “tumor” refers to all neoplastic cell growth and proliferation, whether malignant or benign, and all pre-cancerous and cancerous cells and tissues, and particularly to an abnormal growth of cells or tissues of the malignant type. The “tumor” may be comprised of at least one cell and/or tissue.

As used herein, the term “metastasis” refers to the spread to other locations in the body, for example to another non-adjacent organ or part of an organ.

As used herein, the term “treatment” includes “therapeutic treatment” for example a curative treatment as well as “prophylactic treatment” either preventing or inhibiting the development of cancer or delaying the onset of a pre-clinically evident stage of cancer.

The term “therapeutically effective amount” is used herein to mean an amount or dose sufficient to modulate, e.g., decrease the level of Trim71 activity for example by about 10 percent, preferably by about 50 percent, and more preferably by about 90 percent. Preferably, a therapeutically effective amount is sufficient to cause an improvement in a clinically significant condition in the subject.

The terms “protein”, “polypeptide”, and “peptide” respectively, as used herein refer to synthetic or non-synthetic peptides, as well as purified or modified fragments of natural proteins, native forms or recombinant peptides or proteins. Moreover, the terms “protein”, “polypeptide”, and “peptide” respectively, as used herein refer to pharmacologic acceptable salts, pharmacologic acceptable derivatives and/or conjugates of the respective protein, polypeptide, or peptide.

The term “fragment” of a nucleic acid or amino acid sequence as used herein refers to a nucleic acid or amino acid sequence comprising a subset of the nucleic acid or amino acid sequence according to one of the claimed sequences. The same is applicable to the term “fraction” of the nucleic acid or amino acid sequence.

The term “variant” of a nucleic acid or amino acid sequence as used herein refers to a nucleic acid or amino acid sequence, which is substantially similar in structure and biological activity to a nucleic acid or amino acid sequence according to one of the claimed sequences. Preferably, said term refers to a nucleic acid molecule, which comprises at least one silent single nucleotide mutation (as allowed by the degeneracy of the genetic code).

The term “homologue” of a nucleic acid or amino acid sequence as used herein refers to a nucleic acid or amino acid sequence the sequence of which has one or more nucleotides or amino acids added, deleted, substituted or otherwise chemically modified in comparison to a nucleic acid or amino acid sequence according to one of the claimed sequences, provided that the homologue retains substantially the same binding properties as the latter.

The term “ortholog” as used herein refers to genes or proteins in different species that usually evolved from a common ancestral gene by speciation, normally retaining the same function.

The term “derivative” as used herein, refers to a nucleic acid or amino acid sequence that has similar binding characteristics to a target as a nucleic acid or amino acid sequence according to one of the claimed sequences.

The term “nucleic acid sequence” or “nucleic acid molecule” is intended to indicate any single- or double stranded nucleic acid and/or analogous molecules comprising DNA; cDNA and/or genomic DNA; RNA preferably rRNA, tRNA and/or mRNA; peptide nucleic acid (PNA); locked nucleic acid (LNA) and/or Morpholino.

The term “inhibiting” as used herein, refers to its generally accepted meaning which includes stopping, slowing or ameliorating.

The term “RNA interference” or “RNAi” as used herein, refers to a system within living cells that helps to control which genes are active and how active they are. Involved in RNA interference are small RNA molecules most notably siRNA, miRNA and shRNA. Especially “miRNA” and “siRNA” are the direct products of genes, and can bind to specific other RNAs and either increase or decrease their activity. RNAi is thought to be initiated by long double-stranded RNA molecules, which are processed by an enzyme called “Dicer” into shorter, 21 to 23 nucleotides long dsRNAs denoted small interfering RNAs (siRNAs). siRNA molecules are thought to be incorporated into the RNA-induced silencing complex (RISC), a protein-RNA complex, which acts as a guide for an endogenous nuclease to degrade the target RNA.

The term “microRNA” or “miRNA” as used herein, refers to small single-stranded non coding RNA molecules, which regulate gene expression. Their main function is to down-regulate gene expression. A primary transcript (a pri-miRNA) is processed into a short stem-loop structure called a pre-miRNA and finally into a functional miRNA. Mature miRNA molecules are partially complementary to one or more messenger RNA (mRNA) molecules. The term “microRNA” or “miRNA” as used herein, refers to pri-miRNA, pre-miRNA, mature miRNA, fragments or variants thereof.

The term “small inhibitory RNA” or “siRNA”, also known as “short interfering RNA” or silencing RNA, as used herein, refers to single- or double-stranded RNA molecules that are involved in the RNA interference (RNAi) pathway, where they interfere with the expression of a specific gene.

The term “shRNA” or “small hairpin RNA” or “short hairpin RNA” as used herein, refers to RNA molecules having a hairpin structure that can be used to silence gene expression via RNA interference. Usually, the human U6 promoter (a pol III promoter) is used to drive expression of the shRNA hairpin. This vector is usually passed on to daughter cells, allowing the gene silencing to be inherited. The shRNA hairpin structure is cleaved by the cellular machinery into siRNA.

The term “asRNA” as used herein, refers to anti-sense RNA, i.e. RNA synthesized from the minus strand, or RNA synthesized from other RNAs, including structural RNAs, such as rRNA and tRNA, and mRNA.

The term “hybridization” as used herein is used in reference to the pairing of complementary nucleic acids. The term “stringent conditions” relates to conditions under which a nucleic acid or amino acid sequence will hybridize to its target subsequence, but to no other sequences.

The term “mutation”, as used herein, is meant to refer to changes to the base pair sequence of the genetic material of an organism.

The term “tumor suppressor gene” as used herein refers to a gene that protects a cell from one step on the path to cancer. Tumor suppressor genes, or more precisely, the proteins for which they code, often have a dampening or repressive effect on the regulation of the cell cycle.

The term “oncogene” as used herein refers to a genetic sequence whose expression within a cell provides a function in leading from a normal cell into a tumor cell.

The term “biological sample”, as used herein, refers to a sample obtained from a patient. The sample may be of any biological tissue or fluid. Such samples include, but are not limited to, sputum, blood, serum, plasma, blood cells (e.g., white cells), tissue, core or fine needle biopsy samples, cell-containing body fluids, free floating nucleic acids, urine, peritoneal fluid, and pleural fluid, cerebrospinal fluid, tear fluid, or cells there from. Biological samples may also include sections of tissues such as frozen or fixed sections taken for histological purposes or microdissected cells or extracellular parts thereof. Preferably, the sample is a tissue sample. The biological sample may advantageously comprise cells obtained from a biopsy of a suspected tumour. The biological sample may be processed or treated in someway prior to detecting and/or quantifying Trim71 expression, or the such that an extract of the original sample obtained from the subject is used in the method of the invention.

As a “control” normal cells can be used to detect and/or quantify the expression of Trim71 and/or its mammalian and non mammalian orthologs in a non-cancer cell. The level of expression in a normal cell is considered to be the normal or control level of expression. Thus, in accordance with the present invention, the expression of Trim71 in a cancer cell is typically compared to the control level of Trim71 expression in a normal, healthy control cell, advantageously of the same cell type. Typically, the control has been obtained from a healthy individual.

The term “normal”, as used herein, refers to a cell that is not known to be diseased, and particularly a cell that is not a cancer cell. Typically, such a cell would be obtained from a healthy subject, i.e. a subject that does not have a cancer.

Unless otherwise defined, the technical and scientific terms used herein have the same meaning as commonly understood by one of ordinary skill in the art to which this invention belongs.

According to the present invention a nucleic acid molecule capable of inhibiting the translation of Trim71 and/or its mammalian and non mammalian orthologs is provided, wherein the nucleic acid molecule is selected from the group comprising siRNA, miRNA, shRNA and/or asRNA having a nucleic acid sequence that targets at least 10 contiguous nucleotides of SEQ ID NO: 2, SEQ ID NO: 4, SEQ ID NOs: 8 to 10, SEQ ID NO: 12, SEQ ID NO: 14, SEQ ID NO: 16 and/or RNA equivalents thereof; and/or fragments of the nucleic acid molecule.

Surprisingly, it was found that Trim71 and/or its orthologs can be important in the treatment and diagnosis of cancer.

Trim71 (Lin41) is a tripartite-motif protein or RBCC protein (Ring finger, B-box, coiled-coil domain) which is required for the development of several vertebrate and invertebrate organisms. Tripartite-motif 71 protein or Trim71 is the vertebrate homolog of Lin-41 which belongs to the family of Trim (tripartite-motif) proteins. This is a relatively large group of intracellular factors which harbor common motifs/domains and which have been implicated in a variety of functions. The Trims have also been termed RBCC proteins because the mentioned motifs comprise a RING finger domain, a so-called B-box zinc finger and a coiled-coil sequence element. The molecular function of Trim71 in mammals is unknown. Significantly, Trim71 is strongly regulated by the co-conserved tumor suppressor microRNA let-7. The inventors have discovered a homozygous expression of a mutant allele of the Drosophila ortholog of Trim71, which was termed “Wech”. This Lin41/Trim71 ortholog of the fruitfly is expressed from a conserved single copy gene.

Surprisingly, it was discovered that the Trim71 protein, which is normally down-regulated in the course of embryonic development, and scarcely expressed in adult tissues, is strongly up-regulated and robustly detected in human cancers, especially ovarian and lung cancers and cancers of the kidney. It is hypothesized that Trim71 plays an important role in the control of cellular interactions and of differentiation within the tumor and its microenvironment.

The term “Trim71” refers herein to a family of genes encoding mammalian and non mammalian ortholog proteins. The ortholog of Trim71 in Drosophila is termed “Wech”.

The term “Trim71” or “wech” refers herein to a family of genes encoding orthologue proteins of the RING, B-box and coiled-coil/Tripartite Motif (RBCC/TRIM) protein family whose human ortholog is called Trim71, Lin41 or Lin-41. The mouse (Mus musculus) synonyms are called Trim-71, lin-41, Lin41, Gm1127, Ripply2 or mlin-41. The Drosophila melanogaster synonyms are called Dmel_CG1624, lin-41, or dappled: 1(2)k08815-3. However, the inventors found that the dpld (“dpld” dappled) locus in Drosophila melanogaster does not correspond to CG1624 and therefore renamed CG1624 “wech”. As of the Jun. 24, 2008 and Aug. 7, 2008 the official name of CG1624 in flybase has been changed to wech and given the new CG number “CG42396”.

In other words, the Trim71 or Wech proteins are a group of proteins belonging to the class of the RBCC/TRIM protein family. The Wech proteins are also called Lin-41 protein family.

The GenBank accession number for the human ortholog Trim71 of the Wech gene is NM_{—}001039111 or XM_{—}067369. The GenBank accession number for the Mus musculus ortholog Trim71 of the Wech gene is DQ_{—}005956. The GenBank accession number for the Drosophila melanogaster ortholog of Wech gene is AE013599 (Flybase-ID: FBgn0259745). The databases in which the respective human and mouse genes are listed under the given access number can be accessed over the NCBI server of the US National Library of Medicine at the US National Institute of Health. The Drosophila melanogaster Wech gene is listed under the given access number in Flybase (http://flybase.org), a database of Drosophila Genes & Genomes.

Other genes identified as putative homologs and orthologs of Trim71 or of one another, for example during the construction of HomoloGene, are Trim71 for Pan troglodytes, Trim71 for Bos Taurus, RGD1566388_predicted for Rattus norwegicus, Trim71 for Gallus gallus, AgaP_AGAP005125 for Anopheles gambiae and lin-41 for Caenorhabditis elegans, which accession number for the protein used in sequence comparison is XP_{—}516352.2, XP_{—}610389.3, XP_{—}236676.4, NP_{—}001032352.1, XP_{—}314006.2, and NP_{—}001020998.1, respectively.

The term “Trim71 protein” or “Wech protein” as used herein refers to a protein of the family of proteins of the RING, B-box and coiled-coil/Tripartite Motif (RBCC/TRIM) whose Drosophila ortholog is denoted “Wech” and whose human ortholog is called Trim71, Lin41 or Lin-41. The terms “Trim71 polypeptide” or “Wech polypeptide” and “Trim71 peptide” or “Wech peptide” as used herein refer to peptides and polypeptides of the family of proteins of the RING, B-box and coiled-coil/Tripartite Motif (RBCC/TRIM) whose Drosophila ortholog is denoted “Wech” and whose human ortholog is called Trim71, Lin41 or Lin-41.

The term “human Wech protein” as used herein refers to the proteins of Wech of human origin, denoted “Trim71”.

According to the invention, the nucleic acid molecule capable of inhibiting the translation of Trim71 and/or its mammalian and non mammalian orthologs is selected from the group comprising siRNA, miRNA, shRNA and/or asRNA.

Preferably, the nucleic acid molecule is an RNAi molecule. RNAi technique provides a means for the effective and specific targeting and degradation of Trim71 mRNA in cells in vivo. Suitably, the RNAi molecule is selected from a miRNA, shRNA or siRNA molecule, particularly selected from a shRNA or siRNA molecule. Preferably, the invention provides siRNA molecules, which are usable to specifically reduce or eliminate the expression of Trim71 in tumour cells.

Advantageously, siRNA molecules are able to directly affect Trim71 expression at the mRNA level for example by inhibiting transcription or translation of mRNA or reducing mRNA stability.

Preferably, a preferred nucleic acid molecule capable of inhibiting the translation of Trim71 and/or its mammalian and non mammalian orthologs is a siRNA molecule. Alternatively, a suitable nucleic acid molecule can be a shRNA molecule, which may give rise to siRNA following intracellular processing. Such an approach can be advantageous because it requires the synthesis of a single RNA molecule only. Moreover, the shRNA molecule may be more stable than the respective siRNA. Referring to an shRNA molecule, the loop separating the two complementary regions may be between 3 and 23 nucleotides in length, preferably between 4 and 10 nucleotides, and more preferably between 5 and 7 nucleotides.

The target sequence is advantageously selected from SEQ ID NO: 2, SEQ ID NO: 4, SEQ ID NOs: 8 to 10, SEQ ID NO: 12, SEQ ID NO: 14, SEQ ID NO: 16 or the RNA equivalents thereof. Preferably, siRNA, miRNA, shRNA and/or asRNA molecules target from 10 to 5186 contiguous nucleotides of SEQ ID NO: 2, SEQ ID NO: 4, SEQ ID NOs: 8 to 10, SEQ ID NO: 12, SEQ ID NO: 14, SEQ ID NO: 16 and/or the RNA equivalents thereof.

In a preferred embodiment the mammalian and non mammalian orthologs of Trim71 are selected from the group comprising human Trim71, its murine ortholog Trim71 or its fly ortholog Wech having
a) a nucleic acid sequence selected from the group comprising SEQ ID NO: 2, SEQ ID NO: 4, SEQ ID NOs: 8 to 10, SEQ ID NO: 12, SEQ ID NO: 14, and/or SEQ ID NO: 16, or a fragment, variant, homologue, or derivative thereof having the same function,
b) a nucleic acid sequence having a sequence homology or identity of at least 70, preferably 85%, more preferably 95% with any of the nucleic acid sequences of a),
c) a nucleic acid molecule which comprises, in comparison to the nucleic acid molecule according to a) and/or to b) at least one silent single nucleotide mutation (as allowed by the degeneracy of the genetic code),
d) a nucleic acid molecule which, in comparison to the nucleic acid molecule according to a) and/or to c), is code optimized for a given expression host.

In a more preferred embodiment the nucleic acid sequence of b) has a sequence homology or identity of at least 80, preferably 90%, more preferably 98% with any of the nucleic acid sequences of a).

In some advantageous embodiments, the nucleic acid sequence of the nucleic acid molecule of the invention targets from 10 to 5186 contiguous nucleotides, preferably from 12 to 3138 contiguous nucleotides, of SEQ ID NO: 2, SEQ ID NO: 4, SEQ ID NOs: 8 to 10, SEQ ID NO: 12, SEQ ID NO: 14, SEQ ID NO: 16 and/or their RNA equivalents. Preferably, the nucleic acid sequence targets from 15 to 80 contiguous nucleotides, further preferably from 17 to 29 nucleotides, more preferably from 18 to 25 nucleotides, even more preferably from 19 to 23 nucleotides, and most preferably from 21 to 23 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 and/or the RNA equivalents thereof.

Especially, a siRNA molecule can comprise a nucleic acid sequence of from 17 to 35 nucleotides, preferably from 18 to 28 nucleotides, more preferably from 19 to 23 nucleotides, and most preferably from 21 to 23 nucleotides. In preferred embodiments, a siRNA molecule can comprise from 17 to 35 contiguous nucleotides, preferably from 18 to 28 nucleotides, more preferably from 19 to 23 nucleotides, and most preferably from 21 to 23 nucleotides, which target the appropriate from 17 to 35 contiguous nucleotides, preferably from 18 to 28 nucleotides, more preferably from 19 to 23 nucleotides, and most preferably from 21 to 23 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 or the RNA equivalents thereof.

Especially, a shRNA molecule can comprise a nucleic acid sequence of from 40 to 80 nucleotides, preferably from 42 to 70 nucleotides, more preferably from 45 to 55 nucleotides, and most preferably from 48 to 52 nucleotides. In preferred embodiments, a shRNA molecule can comprise from 40 to 80 nucleotides, preferably from 42 to 70 nucleotides, more preferably from 45 to 55 nucleotides, and most preferably from 48 to 52 nucleotides, which target the appropriate from 40 to 80 nucleotides, preferably from 42 to 70 nucleotides, more preferably from 45 to 55 nucleotides, and most preferably from 48 to 52 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 or the RNA equivalents thereof.

It is within the ability of a person skilled in the art, using known sequence databases to determine a suitable sequence of Trim71 for targeting by nucleic acid molecules especially siRNA. In a particularly advantageous embodiment the target sequence chosen may be unique in an animal genome, and most suitably it is unique in the human genome.

In certain preferred embodiments, the nucleic acid sequence of the nucleic acid molecule of the invention comprises a sequence selected from the group comprising:

| | |
|---|---|
| 5′-CCAGATCTGCTTGCTGTGCAA-3′, | (SEQ ID NO: 22) |
| 5′-TGGGACATACGTGGTGAGTTA-3′, | (SEQ ID NO: 23) |

or the RNA equivalents thereof and/or a sequence complementary thereto.

Preferably, the nucleic acid sequences are the RNA equivalents thereof. Advantageously, the nucleic acid sequence selected from the group comprising SEQ ID NO: 22 and/or SEQ ID NO: 23 can exhibit good inhibition of Trim71.

Advantageously, the nucleic acid molecule, especially a siRNA molecule, for use in accordance with the invention is targeted to a unique sequence of the Trim71 mRNA strand.

In preferred embodiments, the RNA equivalent sequences of the sequences of SEQ ID NO: 22 and/or SEQ ID NO: 23 represent the sense strand of the siRNA molecule.

Two siRNA molecules having the RNA equivalent sequences of the sequences: 5′-CCAGATCTGCTTGCTGTGCAA-3′ (SEQ ID NO: 22) and/or 5′-TGGGACATACGTGGTGAGTTA-3′ (SEQ ID NO: 23) which are given as a DNA sequence were designed to specifically target the human Trim71 mRNA sequence. These siRNA molecules target the human Trim71 mRNA sequence (LOCUS: TRIM71) at nucleotides 90-110 (SEQ ID NO: 22), and 1698-1718 (SEQ ID NO: 23), respectively. These regions provide preferred sequences against which to target siRNA molecules for knock-down of human Trim71.

However, siRNA molecules are usually double stranded molecules and siRNA molecules can comprise two substantially complementary oligonucleotide strands, a sense strand and an antisense strand, which anneal to form a double-stranded region of any suitable length.

Referring to miRNAs, the miRNA can be selected from the group comprising a pri-miRNA, pre-miRNA, mature miRNA or a fragment or variant thereof effective in gene silencing.

Especially, a miRNA molecule can comprise a nucleic acid sequence of from 15 to 40 nucleotides, preferably from 18 to 30 nucleotides, more preferably from 20 to 25 nucleotides, and most preferably from 22 to 24 nucleotides. In preferred embodiments, a miRNA molecule can comprise from 15 to 40 nucleotides, preferably from 18 to 30 nucleotides, more preferably from 20 to 25 nucleotides, and most preferably from 22 to 24 nucleotides, which target the appropriate from 15 to 40 nucleotides, preferably from 18 to 30 nucleotides, more preferably from 20 to 25 nucleotides, and most preferably from 22 to 24 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 or the RNA equivalents thereof.

Antisense nucleic acid sequences are complementary to Trim71 mRNA and thus can hybridise with Trim71 mRNA in-vivo. Antisense nucleic acid sequences may be in the form of single stranded DNA or RNA molecules that hybridise to all or a part of the sequence of Trim71 mRNA. The corresponding cDNA of mammalian and non mammalian orthologs of Trim71 selected from the group comprising human Trim71, its murine ortholog Trim71 and its fly ortholog Wech is given by the group comprising SEQ ID NO: 2, SEQ ID NO: 4, SEQ ID NOs: 8 to 10, SEQ ID NO: 12, SEQ ID NO: 14, and/or SEQ ID NO: 16.

Especially, an asRNA molecule can comprise a nucleic acid sequence of from 50 to 3138 nucleotides, preferably from 60 to 100 nucleotides, more preferably from 70 to 90 nucleotides, and most preferably from 80 to 85 nucleotides. In preferred embodiments, an asRNA molecule can comprise from 50 to 3138 nucleotides, preferably from 60 to 100 nucleotides, more preferably from 70 to 90 nucleotides, and most preferably from 80 to 85 nucleotides, which target the appropriate from 50 to 3138 nucleotides, preferably from 60 to 100 nucleotides, more preferably from 70 to 90 nucleotides, and most preferably from 80 to 85 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 or the RNA equivalents thereof.

The nucleic acid molecules selected from the group comprising siRNA, miRNA, shRNA and/or asRNA, especially antisense oligonucleotides can be used to inhibit expression of Trim71 in target tissues and cells in vivo, or such molecules may be used in an ex vivo treatment, or in an in vitro diagnostic test.

Requirements for the design and synthesis of antisense molecules against a specific target gene, methods for introducing and expressing antisense molecules in a cell, and suitable means for modifying such antisense molecules are known to a person skilled in the art.

The invention encompasses nucleic acid molecules preferably for use in medicine.

In yet another aspect of the invention, the use of a nucleic acid molecule according to the invention, preferably selected from the group comprising:

| | |
|---|---|
| 5′-CCGTGTGCGACCAGAAAGTA-3′, | (SEQ ID NO: 21) |
| 5′-CCAGATCTGCTTGCTGTGCAA-3′, | (SEQ ID NO: 22) |
| 5′-TGGGACATACGTGGTGAGTTA-3′, | (SEQ ID NO: 23) |

or the RNA equivalents thereof and/or a sequence complementary thereto, is provided for preparing a medicament for therapeutic or prophylactic treatment and/or diagnosis of clinical conditions resulting from the detrimental activity of Trim71 and/or its mammalian and non mammalian orthologs.

The invention advantageously encompasses nucleic acid molecules for use in medicine and even more advantageously for use in down-regulating Trim71 expression for the treatment of cancer in a human. Suitably, the nucleic acid molecule usable for therapeutic or prophylactic treatment and/or diagnosis of clinical conditions resulting from the detrimental activity of Trim71 and/or its mammalian and non mammalian orthologs is capable of inhibiting the translation of Trim71 and/or its mammalian and non mammalian orthologs. Preferably, the nucleic acid molecule is selected from the group comprising siRNA, miRNA, shRNA and/or asRNA having a nucleic acid sequence that targets at least 10, preferably from 10 to 5186 contiguous nucleotides of SEQ ID NO: 2, SEQ ID NO: 4, SEQ ID NOs: 8 to 10, SEQ ID NO: 12, SEQ ID NO: 14, SEQ ID NO: 16, and/or their RNA equivalents, and fragments thereof that inhibit the translation of Trim71 and/or its mammalian and non mammalian orthologs. Preferably, the nucleic acid molecule targets from 12 to 3138 contiguous bases, preferably between 21 and 23 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 and/or the RNA equivalents thereof. More preferably, the nucleic acid sequence of the nucleic acid molecule of the invention comprises a sequence selected from the group comprising: 5′-CCGTGTGCGACCAGAAAGTA-3′ (SEQ ID NO: 21), 5′-CCAGATCTGCTTGCTGTGCAA-3′ (SEQ ID NO: 22), 5′-TGGGACATACGTGGTGAGTTA-3′ (SEQ ID NO: 23), or the RNA equivalent thereof and/or a sequence complementary thereto.

The invention further provides evidence of the involvement of Trim71 in cancer as the present invention provides the observation that Trim71 protein is significantly expressed in certain cancer cell types, in comparison to the respective normal cells.

In another aspect of the invention, is provided the use of a nucleic acid molecule according to the invention for preparing a medicament for therapeutic or prophylactic treatment and/or diagnosis of cancer and/or metastasis thereof.

Preferably, the cancer is associated with an up-regulation of Trim71 expression and/or activity in the cancer cells. Preferably, the siRNA, miRNA, shRNA and/or asRNA molecules of the invention are usable in down-regulating the expression of Trim71 for the treatment of cancer.

In a preferred embodiment the cancer is selected from the group comprising thyroid cancer, lung cancer, small cell lung cancer (SCLC), liver cancer, cancers of the kidney, cancers of the atrioventricular node, cancers of the skeletal muscle, skin cancer, salivary gland cancer, ovary cancer, upper gastrointestinal cancers and/or cancers of the nervous system.

The cancer preferably is selected from the group comprising cancers of the atrioventricular node, cancers of the skeletal muscle, skin cancer, salivary gland cancer, ovary cancer, and/or cancers of the nervous system. Even more preferably, the cancer is selected from the group comprising lung cancer, small cell lung cancer (SCLC), cancers of the kidney, and/or ovary cancer.

Preferred upper gastrointestinal cancers are selected from the group comprising pancreas cancer, esophagus cancer, and/or stomach cancer. Preferred cancers of the nervous system are selected from the group comprising cancers of the cingulated cortex, the Medulla oblongata, Temporal lobe, Ciliary ganglion, and/or the Superior cervical ganglion.

For example, nucleic acid molecules for use in therapy may be administered to a patient directly at the site of a tumour, for example, by injection into the cell mass of the tumour, or they can be transcribed from a vector that is transfected into the tumour cells.

The invention also encompasses a pharmaceutical or diagnostic composition comprising a nucleic acid molecule according to the invention.

The pharmaceutical or diagnostic composition advantageously encompasses nucleic acid molecules for use in medicine and advantageously for use in down-regulating Trim71 expression for the treatment of cancer in a human. Preferably, the pharmaceutical or diagnostic composition advantageously encompasses a nucleic acid molecule selected from the group comprising siRNA, miRNA, shRNA and/or asRNA having preferably a nucleic acid sequence that targets at least 10, preferably from 10 to 5186 contiguous nucleotides of SEQ ID NO: 2, SEQ ID NO: 4, SEQ ID NOs: 8 to 10, SEQ ID NO: 12, SEQ ID NO: 14, SEQ ID NO: 16, and/or their RNA equivalents, and fragments thereof that inhibit the translation of Trim71 and/or its mammalian and non mammalian orthologs. Preferably, the nucleic acid molecule targets from 12 to 3138 contiguous bases, preferably between 21 and 23 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 and/or the RNA equivalents thereof. More preferably, the nucleic acid sequence of the nucleic acid molecule of the invention comprises a sequence selected from the group comprising: 5′-CCGTGTGCGACCAGAAAGTA-3′ (SEQ ID NO: 21), 5′-CCAGATCTGCTTGCTGTGCAA-3′ (SEQ ID NO: 22), 5′-TGGGACATACGTGGTGAGTTA-3′ (SEQ ID NO: 23), or the RNA equivalent thereof and/or a sequence complementary thereto.

In yet another aspect of the invention, there is provided a pharmaceutical or diagnostic composition for the therapeutic and/or prophylactic treatment and/or the diagnosis of cancer and/or metastasis thereof comprising a nucleic acid molecule selected from the group comprising siRNA, miRNA, shRNA and/or asRNA having a nucleic acid sequence that targets at least 10 contiguous nucleotides of SEQ ID NO: 2, SEQ ID NO: 4, SEQ ID NOs: 8 to 10, SEQ ID NO: 12, SEQ ID NO: 14, SEQ ID NO: 16, and/or RNA equivalents thereof; and/or fragments of the nucleic acid molecule that inhibit the translation or expression of Trim71 and/or its mammalian and non mammalian orthologs.

The pharmaceutical or diagnostic composition advantageously encompasses nucleic acid molecules that target from 10 to 5186, preferably from 12 to 3138 contiguous bases, more preferably between 21 and 23 contiguous nucleotides of SEQ ID NO: 2, SEQ ID NO: 4, SEQ ID NOs: 8 to 10, SEQ ID NO: 12, SEQ ID NO: 14, and/or SEQ ID NO: 16 and/or the RNA equivalents thereof. More preferably, the nucleic acid sequence of the nucleic acid molecule of the invention comprises a sequence selected from the group comprising:

| | |
|---|---|
| 5′-CCGTGTGCGACCAGAAAGTA-3′, | (SEQ ID NO: 21) |
| 5′-CCAGATCTGCTTGCTGTGCAA-3′, | (SEQ ID NO: 22) |
| 5′-TGGGACATACGTGGTGAGTTA-3′, | (SEQ ID NO: 23) |

or the RNA equivalent thereof and/or a sequence complementary thereto.

Studies of siRNA mediated knock-down of Trim71 in HeLa cells was found to lead to rounding up and detachment of cells from substrate.

Preferably, there is also provided an antibody or antigen binding portion thereof that specifically binds to a Trim71 polypeptide. Preferably, the antibody specifically binds to an antigenic region of Trim71. Preferably, the antibody or antigen binding portion thereof is selected from the group comprising a polyclonal antibody, a monoclonal antibody, a humanised monoclonal antibody derived from a murine monoclonal antibody, and/or a human monoclonal antibody.

Pharmaceutical or diagnostic preparations of the invention can be administered orally, intravenously, topically, or via other standard routes. The pharmaceutical preparations may be in the form of tablets, pills, lotions, gels, liquids, powders, suppositories, suspensions, liposomes, microparticles or other suitable formulations known in the art.

Advantageously, especially the risk of tumor metastasis can be prevented or reduced by administering a nucleic acid molecule capable of inhibiting the translation of Trim71 and/or its mammalian and non mammalian orthologs and/or the pharmaceutical or diagnostic composition comprising an inhibitor of Trim71 and/or its mammalian and non mammalian orthologs.

Another aspect according to the present invention is a pharmaceutical or diagnostic composition for the therapeutic and/or prophylactic treatment and/or for the diagnosis of cancer and/or metastasis thereof comprising an amino acid sequence related to Trim71 and/or its mammalian and non mammalian orthologs and/or a nucleic acid sequence of the gene encoding for Trim71 and/or its mammalian and non mammalian orthologs.

Preferably, the cancer is selected from the group comprising thyroid cancer, lung cancer, small cell lung cancer (SCLC), liver cancer, cancers of the kidney, cancers of the atrioventricular node, cancers of the skeletal muscle, skin cancer, salivary gland cancer, ovary cancer, upper gastrointestinal cancers and/or cancers of the nervous system.

In a more preferred embodiment the cancer is selected from the group comprising cancers of the atrioventricular node, cancers of the skeletal muscle, skin cancer, salivary gland cancer, ovary cancer, and/or cancers of the nervous system. Even more preferably, the cancer is selected from the group comprising lung cancer, small cell lung cancer (SCLC), cancers of the kidney, and/or ovary cancer.

It has been further discovered in accordance with the present invention that in particular cancer selected from the group comprising cancers of the atrioventricular node, cancers of the skeletal muscle, skin cancer, salivary gland cancer, ovary cancer, and/or cancers of the nervous system are susceptible to a treatment and/or diagnosis with an amino acid sequence related to Trim71 and/or its mammalian and non mammalian orthologs and/or a nucleic acid sequence of the gene encoding for Trim71 and/or its mammalian and non mammalian orthologs.

Preferred upper gastrointestinal cancers are selected from the group comprising pancreas cancer, esophagus cancer, and/or stomach cancer.

Preferred cancers of the nervous system are selected from the group comprising cancers of the cingulated cortex, the Medulla oblongata, Temporal lobe, Ciliary ganglion, and/or the Superior cervical ganglion.

In a preferred embodiment the amino acid sequence is related to the human Trim71 protein and/or the nucleic acid sequence of the gene encoding for the human Trim71 protein.

Preferably, treatment and/diagnosis is performed in a human patient, therefore, the protein or peptide of Trim71 and/or its mammalian and non mammalian orthologs and/or the nucleic acid sequence preferably is of human origin. However, even for treatment of humans it might be more preferred that the protein or peptide sequence and/or the nucleic acid sequence used is of non-human origin, for example of murine origin, or even of non-mammal origin, for example of fly, preferably Drosophila melanogaster origin.

In a preferred embodiment the amino acid sequence related to Trim71 and/or its mammalian and non mammalian orthologs is a peptide, polypeptide or protein related to human Trim71, wherein said peptide, polypeptide or protein
a) comprises an amino acid sequence according to SEQ ID NO: 1, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 2, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

In another preferred embodiment the amino acid sequence related to Trim71 and/or its mammalian and non mammalian orthologs is a peptide, polypeptide or protein related to the murine Trim71, wherein said peptide, polypeptide or protein
a) comprises an amino acid sequence according to SEQ ID NO: 3, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 4, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

In yet another preferred embodiment the amino acid sequence related to Trim71 and/or its mammalian and non mammalian orthologs is a peptide, polypeptide or protein related to fly Wech, wherein said peptide, polypeptide or protein
a) comprises an amino acid sequence according to SEQ ID NO: 5 to 7, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 8 to 10, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

It is also preferred that amino acid sequences related to Trim71 and/or its mammalian and non mammalian orthologs for example a protein, a peptide or a polypeptide may be administered since amino acid sequences generally exhibit good stability. Stable proteins or peptides can exhibit longer activity when administered.

Preferably, amino acid sequence related to Trim71 and/or its mammalian and non mammalian orthologs may comprise a partial sequence of the SEQ ID NO: 1.

In a preferred embodiment the amino acid sequence related to Trim71 and/or its mammalian and non mammalian orthologs is a peptide, polypeptide or protein related to human Trim71, wherein said peptide, polypeptide or protein
a) comprises an amino acid sequence according to SEQ ID NO: 11, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 12, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

Advantageously, peptides, polypeptides or proteins related to human Trim71, wherein said peptide, polypeptide or protein comprises an amino acid sequence according to SEQ ID NO: 11, or a fragment, variant, homologue, or derivative thereof as outlined above, will provide better specificity. Moreover, said shorter peptides, polypeptides or proteins comprising an amino acid sequence according to SEQ ID NO: 11, or a fragment, variant, homologue, or derivative thereof may provide lesser side effects on other metabolic processes influenced by Trim71 and/or its mammalian and non mammalian orthologs.

In another preferred embodiment the amino acid sequence related to Trim71 and/or its mammalian and non mammalian orthologs is a peptide, polypeptide or protein related to murine Trim71, wherein said peptide, polypeptide or protein
a) comprises an amino acid sequence according to SEQ ID NO: 13, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 14, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

In yet another preferred embodiment the amino acid sequence related to Trim71 and/or its mammalian and non mammalian orthologs is a peptide, polypeptide or protein related to fly Wech, wherein said peptide, polypeptide or protein
a) comprises an amino acid sequence according to SEQ ID NO: 15, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 16, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

Advantageously, peptides, polypeptides or proteins related to human or murine Trim71 or its ortholog in fly Wech, wherein said peptide, polypeptide or protein comprises an amino acid sequence according to SEQ ID NO: 13, or SEQ ID NO: 15, or a fragment, variant, homologue, or derivative thereof as outlined above, will provide better specificity. Moreover, said shorter peptides, polypeptides or proteins comprising an amino acid sequence according to SEQ ID NO: 13, or SEQ ID NO: 15, or a fragment, variant, homologue, or derivative thereof may provide lesser side effects on other metabolic processes influenced by Trim71 and/or its mammalian and non mammalian orthologs.

In a more preferred embodiment the amino acid sequence of Trim71 and/or its mammalian and non mammalian orthologs is a peptide of eight to twenty, preferably of nine to eighteen, more preferably of ten to sixteen, even more preferably of eleven or twelve, contiguous amino acids of Trim71 and/or its mammalian and non mammalian orthologs.

In more preferred embodiment of the pharmaceutical composition the amino acid sequence related to Trim71 and/or its mammalian and non mammalian orthologs is a peptide selected from the group comprising
KFGEKGTKNGQFNYPW (SEQ ID NO: 17),
CVRAHQRVRLTKDHYI (SEQ ID NO: 18),
LSLSFATEGHEDGQV (SEQ ID NO: 19) and/or
SPDSKEGSNPYKRFVHVF (SEQ ID NO: 20).

It may also be advantageously, to provide nucleic acid sequences of the gene encoding for Trim71 and/or its mammalian and non mammalian orthologs.

In another preferred embodiment, the nucleic acid sequence of the gene encoding for the human Trim71 is
a) a nucleic acid sequence having the nucleotide sequence of SEQ ID NO: 2 or 12, or a fragment, variant, homologue, or derivative thereof,
b) a nucleic acid sequence having a sequence identity of at least 70, preferably 85%, more preferably 95% with any of the nucleic acid sequences of a),
c) a nucleic acid molecule which comprises, in comparison to the nucleic acid molecule according to a) to b) at least one silent single nucleotide mutation (as allowed by the degeneracy of the genetic code), and/or
d) a nucleic acid molecule which, in comparison to the nucleic acid molecule according to a) to c), is code optimized for a given expression host.

In another preferred embodiment, the nucleic acid sequence of the gene encoding for the murine Trim71 is
a) a nucleic acid sequence having the nucleotide sequence of SEQ ID NO: 4 or 14, or a fragment, variant, homologue, or derivative thereof,
b) a nucleic acid sequence having a sequence identity of at least 70, preferably 85%, more preferably 95% with any of the nucleic acid sequences of a),
c) a nucleic acid molecule which comprises, in comparison to the nucleic acid molecule according to a) to b) at least one silent single nucleotide mutation (as allowed by the degeneracy of the genetic code), and/or
d) a nucleic acid molecule which, in comparison to the nucleic acid molecule according to a) to c), is code optimized for a given expression host.

In another preferred embodiment, the nucleic acid sequence of the gene encoding for the fly Wech protein is
a) a nucleic acid sequence having the nucleotide sequence of SEQ ID NO: 8 to 10 or 16, or a fragment, variant, homologue, or derivative thereof,
b) a nucleic acid sequence having a sequence identity of at least 70, preferably 85%, more preferably 95% with any of the nucleic acid sequences of a),
c) a nucleic acid molecule which comprises, in comparison to the nucleic acid molecule according to a) to b) at least one silent single nucleotide mutation (as allowed by the degeneracy of the genetic code), and/or
d) a nucleic acid molecule which, in comparison to the nucleic acid molecule according to a) to c), is code optimized for a given expression host.

Using known sequences, proteins or nucleic acid sequences having the nucleotide sequence of a known sequence, or a fragment, variant, homologue, or derivative thereof may be synthesized using standard chemical peptide synthesis techniques. Where the desired subsequences are relatively short the molecule may be synthesized as a single contiguous polypeptide. Where larger molecules are desired, subsequences can be synthesized separately in one or more units and then fused by condensation of the amino terminus of one molecule with the carboxyl terminus of the other molecule thereby forming a peptide bond.

Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis and affinity purification are described by March et al., 1974 A simplified method for cyanogen bromide activation of agarose for affinity chromatography; Anal. Biochem. July; 60(1):149-52.

Alternatively, proteins or nucleic acid sequences can be synthesized using recombinant DNA methodology. Generally this involves creating a DNA sequence that encodes the fusion protein, placing the DNA in an expression cassette under the control of a particular promoter, expressing the protein in a host, isolating the expressed protein and, if required, renaturing the protein.

Advantageously, the pharmaceutical composition is applied by intravenous, intraarterial, intramuscular, subcutaneous, intraperitoneal, oral, buccal, nasal, rectal, topical, transdermal, epidural, intrathecal application or locally into the tumor.

Preferred embodiments of pharmaceutical or diagnostic formulations comprising amino acid sequence related to Trim71 and/or its mammalian and non mammalian orthologs particularly peptides may comprise a solution of 0.1 M Glycerol. Other preferred embodiments of pharmaceutical formulations may comprise a salt solution, for example a solution of the peptide in Phosphate Buffered Saline (PBS) solution. Preferably, the peptide may be comprised in concentrations of 0.1 M to 1 M.

Another embodiment of the present invention provides a tumor suppressor agent comprising an amino acid sequence related to Trim71 and/or its mammalian and non mammalian orthologs or a nucleic acid sequence of the gene encoding for Trim71 and/or its mammalian and non mammalian orthologs according to the invention.

Surprisingly, it was found that Trim71 and/or its mammalian and non mammalian orthologs can be important in the treatment and/or diagnosis of cancers.

Advantageously, especially the risk of tumor metastasis can be prevented or reduced by administering Trim71 and/or its mammalian and non mammalian orthologs and/or the nucleic acid sequence of the gene encodes for the human ortholog Trim71 and/or its mammalian and non mammalian orthologs.

It is preferred that the amino acid sequence comprised by the tumor suppressor agent is related to the human ortholog Trim71 and/or the nucleic acid sequence of the gene encodes for the human ortholog Trim71.

However, even for treatment of humans it might be more preferred that the tumor suppressor agent comprises a protein and/or the nucleic acid sequence of non-human origin, for example of murine origin, or even of non-mammal origin, for example of fly, preferably Drosophila melanogaster origin.

It is to be understood that the features of the amino acid sequence related to Trim71 and/or its mammalian and non mammalian orthologs and/or a nucleic acid sequence of the gene encoding for the Wech protein according to the pharmacological composition can also be features of the amino acid sequence related to Trim71 and/or its mammalian and non mammalian orthologs and/or a nucleic acid sequence of the gene encoding for Trim71 and/or its mammalian and non mammalian orthologs comprised by the tumor suppressor agent.

Another aspect of the present invention provides the use of an amino acid sequence related to Trim71 and/or its mammalian and non mammalian orthologs and/or a nucleic acid sequence of the gene encoding for Trim71 and/or its mammalian and non mammalian orthologs according to the invention as medication for therapeutic and/or prophylactic treatment and/or for the diagnosis of cancer and/or metastasis thereof.

Advantageously, the amino acid sequence related to Trim71 and/or its mammalian and non mammalian orthologs and/or a nucleic acid sequence of the gene encoding for Trim71 and/or its mammalian and non mammalian orthologs according to the invention can be used to manufacture a medication or a pharmaceutical composition for therapeutic and/or prophylactic treatment or diagnosis of cancer and/or metastasis thereof.

It is preferred that the cancer is selected from the group comprising thyroid cancer, lung cancer, small cell lung cancer (SCLC), liver cancer, cancers of the kidney, cancers of the atrioventricular node, cancers of the skeletal muscle, skin cancer, salivary gland cancer, ovary cancer, upper gastrointestinal cancers, preferably selected from the group comprising pancreas cancer, esophagus cancer, and/or stomach cancer, and/or cancers of the nervous system, preferably selected from the group comprising cancers of the cingulated cortex, the Medulla oblongata, Temporal lobe, Ciliary ganglion, and/or the Superior cervical ganglion.

Preferably, Trim71 and/or its mammalian and non mammalian orthologs and/or the nucleic acid sequence is used as a pharmaceutical agent in humans. Therefore, the protein Trim71 and/or its mammalian and non mammalian orthologs and/or the nucleic acid sequence used preferably is of human origin. However, even for treatment of humans it might be more preferred that the protein Trim71 and/or its mammalian and non mammalian orthologs and/or the nucleic acid sequence used is of non-human origin, for example of murine origin, or even of non-mammal origin, for example of fly, preferably Drosophila melanogaster origin.

It is preferred for the use of an amino acid sequence related to Trim71 and/or its mammalian and non mammalian orthologs and/or a nucleic acid sequence of the gene encoding for the protein Trim71 and/or its mammalian and non mammalian orthologs as medication for therapeutic and/or prophylactic treatment and/or for the diagnosis of cancer and/or metastasis thereof that the amino acid sequence related to Trim71 and/or its mammalian and non mammalian orthologs is a peptide, polypeptide or protein related to human Trim71, wherein said peptide, polypeptide or protein
a) comprises an amino acid sequence according to SEQ ID NO: 1, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 2, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

In another preferred embodiment of the use as medication the amino acid sequence related to Trim71 and/or its mammalian and non mammalian orthologs is a peptide, polypeptide or protein related to murine Trim71, wherein said peptide, polypeptide or protein
a) comprises an amino acid sequence according to SEQ ID NO: 3, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 4, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

In yet another preferred embodiment of the use as medication the amino acid sequence related to Trim71 and/or its mammalian and non mammalian orthologs is a peptide, polypeptide or protein related to fly Wech, wherein said peptide, polypeptide or protein
a) comprises an amino acid sequence according to SEQ ID NO: 5 to 7, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 8 to 10, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

Preferably, usable amino acid sequence related to Trim71 and/or its mammalian and non mammalian orthologs may comprise a partial sequence of the SEQ ID NO: 1, SEQ ID NO: 3 or SEQ ID NO: 5 to 7.

In a preferred embodiment of the use as medication the amino acid sequence related to Trim71 and/or its mammalian and non mammalian orthologs is a peptide, polypeptide or protein related to human Trim71, wherein said peptide, polypeptide or protein
a) comprises an amino acid sequence according to SEQ ID NO: 11, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 12, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

In another preferred embodiment of the use as medication the amino acid sequence related to Trim71 and/or its mammalian and non mammalian orthologs is a peptide, polypeptide or protein related to murine Trim71, wherein said peptide, polypeptide or protein
a) comprises an amino acid sequence according to SEQ ID NO: 13, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 14, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

In yet another preferred embodiment of the use as medication the amino acid sequence related to Trim71 and/or its mammalian and non mammalian orthologs is a peptide, polypeptide or protein related to fly Wech, wherein said peptide, polypeptide or protein
a) comprises an amino acid sequence according to SEQ ID NO: 15, or a fragment, variant, homologue, or derivative thereof,
b) comprises an amino acid sequence having a sequence identity of at least 70, preferably 85%, more preferably 95% with any of the amino acid sequence of a),
c) is encoded by the nucleic acid of SEQ ID NO: 16, or a fragment, variant, homologue or derivative thereof,
d) is encoded by a nucleic acid molecule that is capable of hybridizing to any of the nucleic acid molecules of c) under stringent conditions,
e) is encoded by a nucleic acid molecule having a sequence identity of at least 70, preferably 85%, more preferably 95% with any of the nucleic acid molecules of c) or d),
f) is encoded by a nucleic acid molecule that is capable of hybridizing to the complement of any of the nucleic acid molecules of c) to e) under stringent conditions,
g) is encoded by a nucleic acid molecule which comprises, in comparison to the nucleic acid molecule according to c) to f) at least one silent single nucleotide mutation (as allowed by the degeneracy of the genetic code), and/or
h) is encoded by a nucleic acid molecule which, in comparison to the nucleic acid molecule according to c) to g), is code optimized for a given expression host.

Advantageously, peptides, polypeptides or proteins related to human, murine or fly orthologs of Trim71, wherein said peptide, polypeptide or protein comprises an amino acid sequence according to SEQ ID NO: 11, SEQ ID NO: 13, or SEQ ID NO: 15, or a fragment, variant, homologue, or derivative thereof as outlined above, will provide better specificity. Moreover, said shorter peptides, polypeptides or proteins comprising an amino acid sequence according to SEQ ID NO: 11, SEQ ID NO: 13, or SEQ ID NO: 15, or a fragment, variant, homologue, or derivative thereof may provide lesser side effects on other metabolic processes influenced by Trim71 and/or its mammalian and non mammalian orthologs.

In more preferred embodiment of the use as medication the amino acid sequence related to Trim71 and/or its mammalian and non mammalian orthologs is a peptide selected from the group comprising
KFGEKGTKNGQFNYPW (SEQ ID NO: 17),
CVRAHQRVRLTKDHYI (SEQ ID NO: 18),
LSLSFATEGHEDGQV (SEQ ID NO: 19) and/or
SPDSKEGSNPYKRFVHVF (SEQ ID NO: 20).

Advantageously, peptides selected from the group comprising SEQ ID NO: 17, SEQ ID NO: 18, SEQ ID NO: 19 and/or SEQ ID NO: 20, will provide even better specificity and/or lesser side effects on other metabolic processes influenced by Trim71 and/or its mammalian and non mammalian orthologs.

It is to be understood that the features of the amino acid sequence related to Trim71 and/or its mammalian and non mammalian orthologs and/or a nucleic acid sequence of the gene encoding for Trim71 and/or its mammalian and non mammalian orthologs according to the pharmacological composition can also be features of the amino acid sequence related to Trim71 and/or its mammalian and non mammalian orthologs and/or a nucleic acid sequence of the gene encoding for Trim71 and/or its mammalian and non mammalian orthologs for the use as medication according to the present invention.

It may also be advantageously, to provide nucleic acid sequences of the gene encoding for Trim71 and/or its mammalian and non mammalian orthologs for use of the amino acid sequence related to the Wech protein and/or a nucleic acid sequence of the gene encoding for Trim71 and/or its mammalian and non mammalian orthologs as a medication.

In a preferred embodiment, of the use as medication or a diagnostic composition the nucleic acid sequence of the gene encoding for the human ortholog Trim71 is
a) a nucleic acid sequence having the nucleotide sequence of SEQ ID NO: 2 or 12, or a fragment, variant, homologue, or derivative thereof,
b) a nucleic acid sequence having a sequence identity of at least 70, preferably 85%, more preferably 95% with any of the nucleic acid sequences of a),
c) a nucleic acid molecule which comprises, in comparison to the nucleic acid molecule according to a) to b) at least one silent single nucleotide mutation (as allowed by the degeneracy of the genetic code), and/or
d) a nucleic acid molecule which, in comparison to the nucleic acid molecule according to a) to c), is code optimized for a given expression host.

In another preferred embodiment of the use as a medication or a diagnostic composition, the nucleic acid sequence of the gene encoding for the murine ortholog Trim71 is
a) a nucleic acid sequence having the nucleotide sequence of SEQ ID NO: 4 or 14, or a fragment, variant, homologue, or derivative thereof,
b) a nucleic acid sequence having a sequence identity of at least 70, preferably 85%, more preferably 95% with any of the nucleic acid sequences of a),
c) a nucleic acid molecule which comprises, in comparison to the nucleic acid molecule according to a) to b) at least one silent single nucleotide mutation (as allowed by the degeneracy of the genetic code), and/or
d) a nucleic acid molecule which, in comparison to the nucleic acid molecule according to a) to c), is code optimized for a given expression host.

In another preferred embodiment of the use as a medication or a diagnostic composition, the nucleic acid sequence of the gene encoding for the fly Wech protein is
a) a nucleic acid sequence having the nucleotide sequence of SEQ ID NO: 8 to 10 or 16, or a fragment, variant, homologue, or derivative thereof,
b) a nucleic acid sequence having a sequence identity of at least 70, preferably 85%, more preferably 95% with any of the nucleic acid sequences of a),
c) a nucleic acid molecule which comprises, in comparison to the nucleic acid molecule according to a) to b) at least one silent single nucleotide mutation (as allowed by the degeneracy of the genetic code), and/or
d) a nucleic acid molecule which, in comparison to the nucleic acid molecule according to a) to c), is code optimized for a given expression host.

Another embodiment of the present invention provides peptides, pharmacologic acceptable salts, derivatives and/or conjugates thereof selected from the group comprising
CVRAHQRVRLTKDHYI (SEQ ID NO: 18), and/or
SPDSKEGSNPYKRFVHVF (SEQ ID NO: 20).

Advantageously, peptides selected from the group comprising SEQ ID NO: 18 and/or SEQ ID NO: 20, showed better specificity and/or lesser side effects on other metabolic processes influenced by Trim71 and/or its mammalian and non mammalian orthologs.

Furthermore, the present invention is directed to nucleic acid molecules or amino acid sequence which are fused to one or more further functional components. The present invention also relates to a fusion molecule comprising a nucleic acid molecule or an amino acid sequence according to the invention and at least one functional component being selected from the group comprising binding domains, receptors, antibodies, regulation domains, pro-sequences, and functional fragments thereof, polyethylenglycols, carbohydrates, lipids, fatty acids, nucleic acids, metals, metal chelate, and functional fragments or derivatives thereof.

The present invention also relates to a vector comprising the nucleic acid molecules, a host cell comprising the vector or comprising the nucleic acid molecules. Preferably, the host cell is selected from the group comprising Escherichia coli, Bacillus subtilis, Saccharomyces cerevisiae, Pichia pastonis, Chinese Hamster Ovary (CHO) and/or Baby Hamster Kidney (BHK) cell lines.

It was discovered that the Trim71 protein is robustly detected in human cancers, especially ovarian and lung cancer and cancers of the kidney. Accordingly, a cancer in a subject may be detected and/or diagnosed by detecting and/or quantifying expression levels especially protein expression levels of Trim71 in a biological sample obtained from the subject.

The present invention also relates to methods for detecting and/or diagnosing the presence of cancer in a subject, comprising detecting and/or quantifying the expression of Trim71 in a biological sample obtained from said subject. The means of detection and/or quantification may involve detecting and/or quantifying a Trim71 polypeptide or a portion or fragment thereof.

Thus, the invention relates to a method of detecting and/or diagnosing cancer, the method comprising the steps of:
providing a biological sample from a patient;
detecting and/or quantifying the expression level of Trim71 in the biological sample, preferably by measuring the expression level of a protein according to SEQ ID NO: 1, SEQ ID NO: 3, SEQ ID NOs: 5 to 7, SEQ ID NO: 11, SEQ ID NO: 13, SEQ ID NO: 15 or the polynucleotide of Trim71 and/or its mammalian and non mammalian orthologs according to SEQ ID NO: 2, SEQ ID NO: 4, SEQ ID NOs: 8 to 10, SEQ ID NO: 12, SEQ ID NO: 14, SEQ ID NO: 16 and/or RNA equivalents thereof; and
comparing the level of Trim71 in the biological sample with that in a control sample, wherein a different expression level of Trim71 in the biological sample compared to that in the control sample indicates the presence of cancer in the patient.

Typically, expression of Trim71 at a level in excess of the level in the control sample is indicative of the presence of cancer. The control sample typically comprises corresponding non-cancerous cells from a healthy individual.

Also encompassed within the scope of the invention is a method of treating cancer in a subject, the method comprising administering to the subject a therapeutically effective amount of a nucleic acid molecule capable of inhibiting the translation of Trim71 and/or its mammalian and non mammalian orthologs, wherein the nucleic acid molecule is selected from the group comprising siRNA, miRNA, shRNA and/or asRNA having a nucleic acid sequence that targets at least 10 contiguous nucleotides of SEQ ID NO: 2, SEQ ID NO: 4, SEQ ID NOs: 8 to 10, SEQ ID NO: 12, SEQ ID NO: 14, SEQ ID NO: 16 and/or RNA equivalents thereof; and/or fragments of the nucleic acid molecule.

Further features of the present invention will become apparent from the figures and examples, which, in an exemplary fashion, show preferred embodiments of the present invention. However, these examples should by no means be understood as to limit the scope of the invention.

Referring to the figures shows:

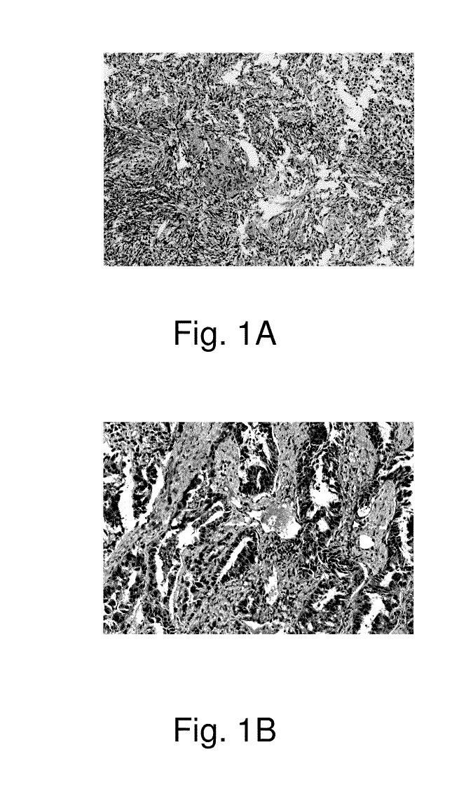

FIG. 1 TRIM71 protein expression in normal (A) and cancerous (B) ovarian tissue.

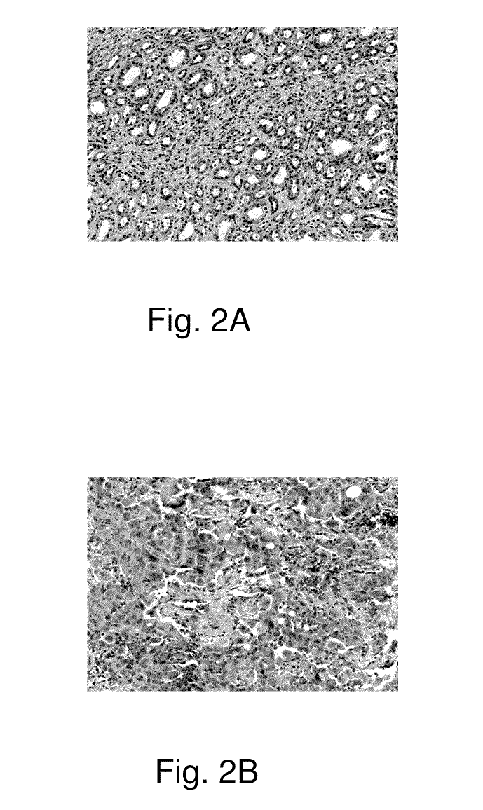

FIG. 2 TRIM71 protein expression in normal (A) and cancerous (B) kidney cells.

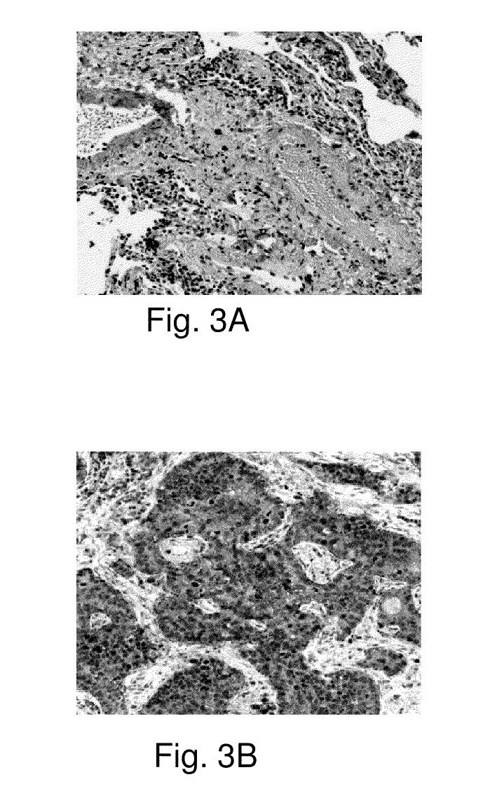

FIG. 3 TRIM71 protein expression in normal (A) and cancerous (B) lung tissue.

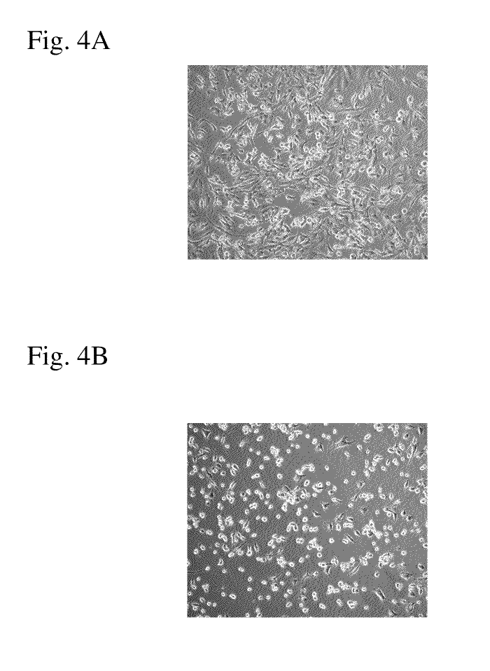

FIG. 4 HeLa cells transfected with control siRNA (A) and (B) in HeLa cells transfected with siRNA targeting TRIM71.

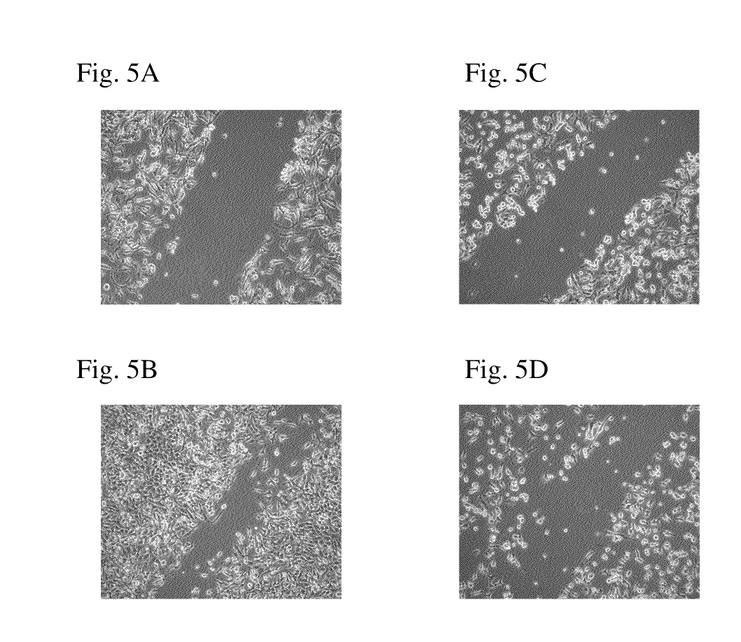

FIG. 5 “scratch” wound healing in HeLa cells transfected with control siRNA (A, B) and in HeLa cells transfected with siRNA targeting TRIM71 (C, D).

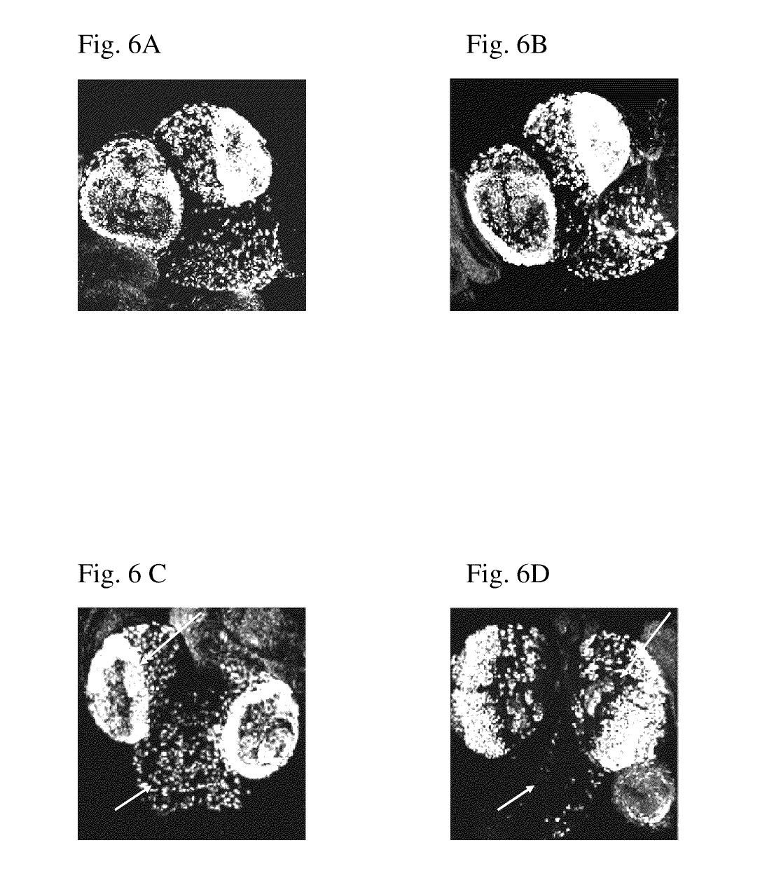

FIG. 6 BrdU positive cells after overexpression of wechGFP in normal larval brain (A, B) and after RNAi knock-down of wech in larval brain (C, D).

### EXAMPLE 1

### Studies of the Expression of TRIM71 Protein in Human Cancer Tissues

Expression of the TRIM71 protein was analyzed in human cancer tissues of ovarian carcinoma, kidney carcinoma, and lung carcinoma by Immunohistochemistry.

Human tumor tissue and control samples for immunohistochemistry were fixed in Carnoy's Fixative for 2 hours or in formalin over night, dehydrated, and embedded in paraffin. Serial 4 micron sections were cut on a microtome (Leica).

Immunohistochemistry was effected on serial sections by an immunoperoxidase technique, using streptavidin-horse-radish-peroxidase and AEC substrate (streptavidin/horseradish peroxidase, Dako, Chem-Mate Detektionskit, Dako).

Tissue samples were deparaffinized and rinsed with PBS. Following, tissue sections were blocked using 200 μl of a blocking solution (Chem-Mate Detektionskit, Dako) for 1 hour. Hybridoma supernatant of a monoclonal primary rat anti-TRIM71 antibody (anti-TRIM71 5B7, GSF, Munich) recognizing peptide sequence SEQ ID NO:17 was diluted 1:10 in blocking solution and tissues were incubated with 150 μl antibody solution over night at 4° C. After washing with PBS, tissues were incubated with 150 μl solution containing secondary anti rat antibody diluted 1:400 in blocking solution for 1 hour at room temperature. Following, tissues were incubated with 3% H₂O₂ for 10 minutes to avoid endogenous peroxidase activity.

Next, the tissue was incubated for 60 minutes with 150 μl streptavidin/horseradish peroxidase (Dako, Chem-Mate Detektion Kit, Dako). Sections were stained with chromogen (Chem-Mate Detektion Kit, Dako) for 10 minutes and counterstained with Mayer's haemalaune. As a control for the specificity of immuno staining, tissues were processed as above, except that non-immune serum was substituted for the primary antibody. Tissues were examined under the light microscope.

FIG. 1 shows TRIM71 protein expression in ovarian tissue. In particular FIG. 1 shows that TRIM71 protein expression, depicted in dark grey, was massively up-regulated in cancerous (1B) tissue compared to normal tissue (1A).

FIG. 2 shows TRIM71 protein expression in kidney tissue. In particular it shows that TRIM71 protein expression, depicted in dark grey, was about five-fold up-regulated in cancerous (2B) tissue compared to normal tissue (2A).

FIG. 3 shows TRIM71 protein expression in lung tissue. In particular it shows that TRIM71 protein expression, depicted in dark grey, was about ten-fold up-regulated in cancerous (3B) tissue compared to normal tissue (3A).

Thus, it was found that TRIM71 protein was significantly expressed in ovarian (FIG. 1), kidney cell (FIG. 2) and lung cell carcinoma (FIG. 3). It thus appears that TRIM71 expression is robustly detected in human cancer tissue but not in normal tissue. Furthermore, importantly it was found that the protein TRIM71 was up-regulated in human cancer tissue.

### EXAMPLE 2

siRNA Knockdown of TRIM71 in HeLa Cells

siRNA transfection technology which accomplished efficient knock-down of the TRIM71 mRNA in the human HeLa cell line, a epithelial cell line derived from a cervical carcinoma, was used to study the effect of TRIM71 siRNA knockdown in human cancer cells. The employed siRNA construct comprised the RNA equivalent of SEQ ID NO: 21.

siRNA Constructs

The siRNA target sequences given as cDNA in SEQ ID NOs: 21 was used to design the siRNA sequences for use in the knockdown of TRIM71 in HeLa cells. The double stranded siRNA molecule comprising the RNA equivalent of SEQ ID NO: 21 as a sense strand and the corresponding anti-sense strand was purchased (Qiagen).

siRNA Transfection

Standard tissue culture dishes (Nunc) were coated with commercially available fibronectin (Harbour Bio-Products) at 50 micrograms/ml in PBS for 1 h at 37° C. One day before transfection 1.5×10⁶ Hela cells were plated on fibronectin coated 6 well plates in 2 ml of growth medium with 10% fetal calf serum (DMEM, Invitrogen) without antibiotics. At the time of transfection the cells were 30-50% confluent.

For each transfection sample, oligomer-Lipofectamine 2000 complexes were prepared according to the manufacturers instructions as follows: 320 pmol siRNA (Qiagen) were diluted in 250 μl Opti-MEM Reduced Serum Medium without serum (Invitrogen). 5 μl Lipofectamine 2000 (Invitrogen) were diluted in 250 μl Opti-MEM Reduced Serum Medium and incubated for 5 minutes at room temperature. After the 5 minute incubation, the diluted siRNA was combined with the diluted Lipofectamine 2000 and further incubated for 20 minutes at room temperature.

After incubation the oligomer-Lipofectamine 2000 complexes were added to each well containing cells and 2 ml medium. The cells were then incubated at 37° C. for 72 hours and were subsequently evaluated by microscopical analysis.

Parallel experiments were also conducted in which a control siRNA that does not target TRIM71 was used as a negative control.

It was observed that following transfection with siRNA targeting TRIM71, cells were rounding up, and finally detached from the substrate, as can be seen in FIG. 4A showing the control cells and FIG. 4B showing HeLa cells transfected with siRNA targeting TRIM71. This shows that siRNA mediated knock-down of TRIM71 protein expression in human HeLa cells results in a strong loss of productive anchorage, detachment of the cells and in subsequent cell death.

### EXAMPLE 3

“Scratch” Wound Assay after siRNA Knockdown of TRIM71 in HeLa Cells

HeLa cells were plated on fibronectin coated 6 well plates and transfected with siRNA targeting TRIM71 as described in Example 2.

After incubation the oligomer-Lipofectamine 2000 complexes and incubation at 37° C. for 72 hours the cell layer was scratched (wounded) with the help of a standard pipette tip. As control type HeLa cells transfected with control siRNA were plated on fibronectin coated plates under identical conditions. The cells were then incubated at 37° C. for another 24 hours and were subsequently evaluated by microscopical analysis.

It was observed that siRNA mediated knock-down of TRIM71 resulted in an impaired “scratch” wound healing, while control cells closed the “wound” through onset of migration and proliferation within 24-48 h. As can be seen in FIG. 5B taken after 24 hours compared to FIG. 5A, control cells showed migration and proliferation into the scratch mark within 24 hours, while HeLa cells transfected with siRNA targeting TRIM71 results in an impaired “scratch” wound healing, as can be seen in FIG. 5D taken after 24 hours compared to FIG. 5C.

These experiments show that TRIM71 has a significant role in proliferation of cancer cells and that transfection with siRNA targeting TRIM71 can provide a useful therapeutic tool in cancer therapy.

### EXAMPLE 4

Measurements of dividing cells in mutant third instar larval wech brains:

### Fly Strains

The generation of inducible RNAi fly strains was performed as described by Lee and Carthew (Lee, S, and Carthew, R. W., Making a better RNAi vector for Drosophila: use of intron spacers. Methods 30, 322-9, 2003). In brief, a 785 bp fragment (ATCGCAACAGTCCGCTGTCCTCCAACCACTCGATCGTGTCCTTGCCCACGCCCATTG GAGCCTCGCCCACGGGTGGCAGCTCGGTAAATGCACAGACTCCGCCCAGCGGCAAC TTTATCTGCGACATACACAACGAGATGTTGCGCTACGTATGTGACTACTGCCGGAAA TTGGTGTGTCAGTGCTGCACACTGCACGAGCACAAGGAGCACAGCTACGCGTCCAT CCAAAGCTTTATGGTGGGCTCGAAGGAGAAGCTGGAGGGCGCCATTGAGAGCAGCC AGGTGGGCACGCGCTGCATTAAGAGCAGCATTGACAAAGCGTTGGCCTTCATCCGG CTTATCGAGCGCAATTGCAGCGAGCTGAGCGATAATATACGCAAGGCATTCCGTCA GTTTATCATTGCCATCGAGGACCGCGAGCGTTTCCTCCTGGACTTTGTGGAAAAGCT CCGCCAGCGTCGTCTGGCCATCCTACACGATCAGATGGCAGGCTTAAAGTCTGCTCT CGCCGGACTCTCCGAAACGTCCGATATGCTTAGCAAGGTGGCGGACAATGCCTGCA ACATGGACCAGATTGAAATTGCCATGAAGTTGACCAATGGGCAGAGGCAGATGGAG CAGTTTGCGGGCATATATAAGGACCTGCAGCCAAAACAGGAAGTCTTTGCCTTCGCA CCACCAGATTACAGCCTGCTACAGGATATCCGCAACCAGGGTGGCGTTATCCTGGTG GACGACAAGAACTTGCCCATCGTCTCTAGCAGCAACGGAATTGTGCCGAGCG, SEQ ID NO: 24) of exon 3 (bp 225 to 1010) of the wech gene was amplified by PCR and cloned in two different orientations into the pWiz vector (gift of R. W. Carthew). Recombinats with the so-called “tail to tail” orientation were selected to generate transgenic flies. To generate a wech-GFP fusion construct, a full-length wech cDNA (Genbank accession number BT010087) was cloned via EcoRI/ApaI restriction sites in frame with the eGFP coding sequence of the pMJ-Green vector (gift from K. Willecke, Bonn). The created wech-GFP fusion construct was excised using the EcoRI/NotI restriction sites and cloned into the pUAST vector (gift of N. Perrimon; see: Brand, A. and Perrimon, N. 1993; Targeted gene expression as a means of altering cell fates and generating dominant phenotypes; Development 118, 401-15) to produce UAS-wech-GFP transgenic flies. The nucleotide sequence of all constructs was confirmed by sequencing.

For overexpression experiments, the inducible heat-shock Gal4 driver line was used to overexpress wech-GFP, wech-RNAi and as a control the wildtype strain OrgeonR at certain time points to generate mutant individuals. In general, males of the different genotypes were crossed to virgins of the heat-shock Gal4 driver line and incubated at 25° C. After 24 h, a 2 h heat-shock was applied to induce the overexpression or knock down of wech. This procedure was performed every day until preparation of the third instar larval brains (−96 h after egg deposition).

The dissection of the mutant larval brains was carried out in prewarmed (room temperature) Schneider's media (Invitrogen). The mutant brains were collected and incubated in 500 μl Schneider's media with 50 μl BrdU (Sigma B5002; concentration: 1 mg/ml) rotating for 2 h. The tissue was rinsed three times with Schneider's media and then incubated for 15 min with Schneider's media. Afterwards, the tissue was rinsed three times with PBS and washed for 15 min in PBS. The following fixation was carried out with 4% paraformaldehyde in PBS for 15 min at room temperature. Afterwards the tissue was washed three times for 5 min with PBT (PBS/0,1% TritonX-100) and then incubated for 30 min in PBT/2N HCl (5 parts PBT and 1 part conc. HCl). The tissue was now blocked in PBTN (PBT+10% donkey-serum) for 30 min at room temperature. The primary BrdU-antibody (Becton-Dickinson #555627) was diluted 1:100 in PBTN and incubated with the tissue overnight at 4° C. The tissue was washed three times 10 min with PBT and then incubated with the secondary antibody donkey anti-mouse Alexa488 (Molecular Probes) for 2 h at room-temperature, light protected. Afterwards, the tissue was washed four times with PBT for 10 min (light protected) and finally dissected and mounted in gel mount (Biomeda, Foster City, USA).

Analysis of the mutant brains was carried out using a Zeiss LSM710 confocal microscope with Zen-software. Processing of the pictures was performed using Adobe Photoshop CS2 software.

It was observed that over expression of WechGFP resulted in more BrdU positive cells in larval brain, as can be seen in FIG. 6B showing larval brain expressing hs-wechGFP compared to the control of FIG. 6A, while a RNAi knock-down of wech in larval brain resulted in less BrdU positive cells, as can be seen in FIG. 6D compared to the control of FIG. 6C.

This shows that wech activates cell division in larval brain and functions as an oncogene.

### EXAMPLE 5

### A Pharmaceutical Composition Comprising a Human TRIM71 Peptide

An aqueous solution of 0.1 M Glycerol of pH 2.4 comprising 1 M of a peptide having the following sequence as set forth in SEQ ID NO: 11 LCRPWGVSVDKEGYIIVADRSNNRIQVFKPCGAFHHKFGTLGSRPGQFDRPAGVACDAS RRIVVADKDNHRIQIFTFEGQFLLKFGEKGTKNGQFNYPWDVAVNSEGKILVSDTRNHR IQLFGPDGVFLNKYGFEGALWKHFDSPRGVAFNHEGHLVVTDFNNHRLLVIHPDCQSA RFLGSEGTGNGQFLRPQGVAVDQEGRIIVADSRNHRVQMFESNGSFLCKFGAQGSGFGQ MDRPSGIAITPDGMIVVVDFGNNRILVF (SEQ ID NO: 11)

### EXAMPLE 6

### A Pharmaceutical Composition Comprising a Murine Trim71 Peptide

An aqueous solution of 0.1 M Glycerol of pH 2.4 comprising 1 M of a peptide having the following sequence as set forth in SEQ ID NO: 13 LCRPWGVSVDKEGFIIVADRSNNRIQVFKPCGSFHHKFGTLGSRPGQFDRPAGVACDAS RRIIVADKDNHRIQIFTFEGQFLLKFGEKGTKNGQFNYPWDVAVNSEGKILVSDTRNHRI QLFGPDGVFLNKYGFEGSLWKHFDSPRGVAFNNEGHLVVTDFNNHRLLVIHPDCQSAR FLGSEGSGNGQFLRPQGVAVDQEGRIIVADSRNHRVQMFEANGSFLCKFGAQG SGFGQMDRPSGIAVTPDGLIVVVDFGNNRILIF (SEQ ID NO: 13).

### EXAMPLE 7

### A Pharmaceutical Composition Comprising a Trim71 Peptide

An aqueous solution of Phosphate Buffered Saline (PBS) buffer comprising 130 mM NaCl, 7 mM Na₂HPO₄, 3 mM NaH₂PO4 of pH 7.2 comprising 0,1 M of a peptide having the following sequence as set forth in SEQ ID NO: 17: KFGEKGTKNGQFNYPW (SEQ ID NO: 17).

### EXAMPLE 8

### A Pharmaceutical Composition Comprising a Trim71 Peptide

An aqueous solution of Phosphate Buffered Saline (PBS) buffer comprising 130 mM NaCl, 7 mM Na₂HPO₄, 3 mM NaH₂PO4 of pH 7.2 comprising 1 M of a peptide having the following sequence as set forth in SEQ ID NO: 18: CVRAHQRVRLTKDHYI (SEQ ID NO: 18).

### EXAMPLE 9

### A Pharmaceutical Composition Comprising a Wech Peptide

An aqueous solution of 0.1 M Glycerol of pH 2.4 comprising 1 M of a peptide having the following sequence as set forth in SEQ ID NO: 15 VSRPWGLCVDKMGHVLVSDRRNNRVQVFNPDGSLKFKFGRKGVGNGEFDLPAGICVD VDNRIIVVDKDNHRVQIFTASGVFLLKFGSYGKEYGQFQYPWDVAVNSRRQIVVTDSRN HRIQQFDSEGRFIRQIVFDNHGQTKGIASPRGVCYTPTGNIIVSDFDNHCLYLIDPDINDIL SVKGHEGSGFHEFNRPSGLCCDDEGRIIVADSKNQRILVFNQNLDFMWDIEVRPSINPLM PPTLDEKDRTCDVAIMPDGRIVFLIELSPDSKEGSNPYKRFVHVF (SEQ ID NO: 15).

### EXAMPLE 10

### A Pharmaceutical Composition Comprising a Trim71 Peptide

An aqueous solution of Phosphate Buffered Saline (PBS) buffer comprising 130 mM NaCl, 7 mM Na₂HPO₄, 3 mM NaH₂PO4 of pH 7.2 comprising 0,1 M of a peptide having the following sequence as set forth in SEQ ID NO: 19: LSLSFATEGHEDGQV (SEQ ID NO: 19).

### EXAMPLE 11

### A Pharmaceutical Composition Comprising a Trim71 Peptide

An aqueous solution of Phosphate Buffered Saline (PBS) buffer comprising 130 mM NaCl, 7 mM Na₂HPO₄, 3 mM NaH₂PO4 of pH 7.2 comprising 0,1 M of a peptide having the following sequence as set forth in SEQ ID NO: 20: SPDSKEGSNPYKRFVHVF (SEQ ID NO: 20).

Unless otherwise indicated, the methods of the present invention used conventional techniques of chemistry, molecular biology, microbiology, recombinant DNA and immunology, which are within the capabilities of a person of ordinary skill in the art. Such techniques are explained in the literature, for example: J. Sambrook, E. F. Fritsch, and T. Maniatis, 1989, Molecular Cloning: A Laboratory Manual, Second Edition, Books 1-3, Cold Spring Harbor Laboratory Press.

It is to be understood that this invention is not limited to the particular features of the composition described as such means and methods may vary. It is also to be understood that the terminology used herein is for purposes of describing particular embodiments only, and is not intended to be limiting. It must be noted that, as used in the specification and the appended claims, the singular forms “a” “an” and “the” include singular and/or plural referents unless the context clearly dictates otherwise. It is also to be understood that plural forms include singular and/or plural referents unless the context clearly dictates otherwise. It is moreover to be understood that, in case parameter ranges are given which are delimited by numeric values, the ranges are deemed to include these limitation values.

The particular combinations of elements and features in the above detailed embodiments are exemplary only. As those skilled in the art will recognize, variations, modifications, and other implementations of what is described herein can occur to those of ordinary skill in the art without departing from the spirit and the scope of the invention as claimed. Accordingly, the foregoing description is by way of example only and is not intended as limiting. The invention's scope is defined in the following claims and the equivalents thereto. Furthermore, reference signs used in the description and claims do not limit the scope of the invention as claimed.

1. A nucleic acid molecule capable of inhibiting the translation of Trim71 and/or its mammalian and non mammalian orthologs, wherein the nucleic acid molecule is selected from the group comprising siRNA, miRNA, shRNA and/or asRNA having a nucleic acid sequence that targets at least 10 contiguous nucleotides of SEQ ID NO: 2, SEQ ID NO: 4, SEQ ID NOs: 8 to 10, SEQ ID NO: 12, SEQ ID NO: 14, SEQ ID NO: 16 and/or RNA equivalents thereof; and/or fragments of the nucleic acid molecule.

2. The nucleic acid molecule according to claim 1, characterized in that the mammalian and non mammalian orthologs of Trim71 are selected from the group comprising human Trim71, its murine ortholog Trim71 or its fly ortholog Wech having:
a) a nucleic acid sequence selected from the group comprising SEQ ID NO: 2, SEQ ID NO: 4, SEQ ID NOs: 8 to 10, SEQ ID NO: 12, SEQ ID NO: 14, and/or SEQ ID NO: 16, or a fragment, variant, homologue, or derivative thereof having the same function,
b) a nucleic acid sequence having a sequence homology of at least 70, preferably 85%, more preferably 95% with any of the nucleic acid sequences of a),
c) a nucleic acid molecule which comprises, in comparison to the nucleic acid molecule according to a) and/or to b) at least one silent single nucleotide mutation (as allowed by the degeneracy of the genetic code),
d) a nucleic acid molecule which, in comparison to the nucleic acid molecule according to a) and/or to c), is code optimized for a given expression host.

3. The nucleic acid molecule according to claims 1, characterized in that the nucleic acid molecule comprises a sequence selected from the group comprising:

| | |
|---|---|
| 5′-CCAGATCTGCTTGCTGTGCAA-3′, | (SEQ ID NO: 22) |
| 5′-TGGGACATACGTGGTGAGTTA-3′, | (SEQ ID NO: 23) |

or the RNA equivalents thereof and/or a sequence complementary thereto.

4. Use of a nucleic acid molecule according to claim 1, preferably selected from the group comprising:

| | |
|---|---|
| 5′-CCGTGTGCGACCAGAAAGTA-3′, | (SEQ ID NO: 21) |
| 5′-CCAGATCTGCTTGCTGTGCAA-3′, | (SEQ ID NO: 22) |
| 5′-TGGGACATACGTGGTGAGTTA-3′, | (SEQ ID NO: 23) |

or the RNA equivalents thereof and/or a sequence complementary thereto for preparing a medicament for therapeutic or prophylactic treatment and/or diagnosis of clinical conditions resulting from the detrimental activity of Trim71 and/or its mammalian and non mammalian orthologs.

5. Use of a nucleic acid molecule according to claim 1 for preparing a medicament for therapeutic or prophylactic treatment and/or diagnosis of cancer and/or metastasis thereof.

6. Use of a nucleic acid molecule according to claim 5, characterized in that the cancer is selected from the group comprising thyroid cancer, lung cancer, small cell lung cancer (SCLC), liver cancer, cancers of the kidney, cancers of the atrioventricular node, cancers of the skeletal muscle, skin cancer, salivary gland cancer, ovary cancer, upper gastrointestinal cancers and/or cancers of the nervous system.

7. A pharmaceutical or diagnostic composition comprising a nucleic acid molecule according to claim 1.

8. A pharmaceutical or diagnostic composition for the therapeutic and/or prophylactic treatment and/or the diagnosis of cancer and/or metastasis thereof comprising a nucleic acid molecule selected from the group comprising siRNA, miRNA, shRNA and/or asRNA having a nucleic acid sequence that targets at least 10 contiguous nucleotides of SEQ ID NO: 2, SEQ ID NO: 4, SEQ ID NOs: 8 to 10, SEQ ID NO: 12, SEQ ID NO: 14, SEQ ID NO: 16, and/or RNA equivalents thereof; and/or fragments of the nucleic acid molecule, that inhibit the translation of Trim71 and/or its mammalian and non mammalian orthologs.

9. A pharmaceutical or diagnostic composition for the therapeutic and/or prophylactic treatment and/or for the diagnosis of cancer and/or metastasis thereof comprising an amino acid sequence related to Trim71 and/or its mammalian and non mammalian orthologs and/or a nucleic acid sequence of the gene encoding for Trim71 and/or its mammalian and non mammalian orthologs.

10. Tumor suppressor agent comprising an amino acid sequence related to Trim71 and/or its mammalian and non mammalian orthologs and/or a nucleic acid sequence of the gene encoding for Trim71 and/or its mammalian and non mammalian orthologs according to claim 1.

11. Use of an amino acid sequence related to Trim71 and/or its mammalian and non mammalian orthologs and/or a nucleic acid sequence of the gene encoding for Trim71 and/or its mammalian and non mammalian orthologs according to claim 1 as medication for therapeutic and/or prophylactic treatment and/or for the diagnosis of cancer and/or metastasis thereof.

12. Peptides, pharmacologic acceptable salts, derivatives and/or conjugates thereof selected from the group comprising:

| | |
|---|---|
| CVRAHQRVRLTKDHYI, | (SEQ ID NO: 18) |
| and/or | |
| SPDSKEGSNPYKRFVHVF. | (SEQ ID NO: 20) |

13. A fusion molecule comprising a nucleic acid molecule or an amino acid sequence according to claim 1 and at least one functional component being selected from the group comprising binding domains, receptors, antibodies, regulation domains, pro-sequences, and functional fragments thereof, polyethylenglycols, carbohydrates, lipids, fatty acids, nucleic acids, metals, metal chelate, and functional fragments or derivatives thereof.

14. A method of detecting and/or diagnosing cancer, the method comprising the steps of:
providing a biological sample from a patient;
detecting and/or quantifying the expression level of Trim71 in the biological sample, preferably by measuring the expression level of a protein according to SEQ ID NO: 1, SEQ ID NO: 3, SEQ ID NOs: 5 to 7, SEQ ID NO: 11, SEQ ID NO: 13, SEQ ID NO: 15 or the polynucleotide of Trim71 and/or its mammalian and non mammalian orthologs according to SEQ ID NO: 2, SEQ ID NO: 4, SEQ ID NOs: 8 to 10, SEQ ID NO: 12, SEQ ID NO: 14, SEQ ID NO: 16 and/or RNA equivalents thereof; and
comparing the level of Trim71 in the biological sample with that in a control sample,
wherein a different expression level of Trim71 in the biological sample compared to that in the control sample indicates the presence of cancer in the patient.

15. A method for treating cancer in a subject, the method comprising administering to the subject a therapeutically effective amount of a nucleic acid molecule capable of inhibiting the translation of Trim71 and/or its mammalian and non mammalian orthologs, wherein the nucleic acid molecule is selected from the group comprising siRNA, miRNA, shRNA and/or asRNA having a nucleic acid sequence that targets at least 10 contiguous nucleotides of SEQ ID NO: 2, SEQ ID NO: 4, SEQ ID NOs: 8 to 10, SEQ ID NO: 12, SEQ ID NO: 14, SEQ ID NO: 16 and/or RNA equivalents thereof; and/or fragments of the nucleic acid molecule.
